SEQUENCE LISTING (9 sequences)

SEQ ID NO: 1 - DNA, 1755 bp, synthetic construct
  source 1..1755: mol_type = other DNA
gatgcgcacaaatcagaggtggcgcataggttcaaggaccttggagaagaaaacttcaaggctctagtattgatagcatttgcacaatacttacaacagtgtccatttgaagaccacgtaaaactcgttaatgaggtcactgagttcgccaagacctgcgtggccgatgaatcggcggagaattgtgataaaagtcttcacactcttttcggcgacaagctatgcactgtagcgacattgcgagaaacctacggtgagatggcggattgctgtgccaagcaggagcctgagcgcaatgagtgttttttgcagcacaaagatgacaaccccaatcttcccaggttggtgcggcccgaagttgacgttatgtgcacggcgtttcatgacaacgaagagacttttctgaagaagtatctatatgaaatagcacggagacacccatatttttacgcccctgagctgctcttttttgctaagaggtataaggcggcttttacagaatgttgtcaggcagcggataaggccgcgtgcctcttacccaagttggacgaacttcgagatgaagggaaggcgtctagtgccaagcaacgtctcaagtgcgcatcactccagaagttcggagagagagccttcaaagcttgggcggtggcccgactatcccagcgctttccgaaagctgagttcgcagaggtatcgaagctcgtaaccgaccttacgaaagtacatactgaatgttgccatggtgacttgctggagtgcgcggatgacagagctgatctagcgaaatacatatgtgaaaaccaagatagcattagtagcaagctgaaagaatgctgcgaaaaacccttgttggagaagtctcattgcattgctgaagtagaaaatgacgagatgccggcagatcttccctccttagccgcggatttcgtcgagagcaaggacgtttgtaaaaattacgcagaagccaaggatgtgttcctagggatgtttctgtacgagtatgcccgtcgtcacccggattattcagtcgtcctgctgctccggttagccaaaacatacgagactaccttagaaaaatgctgtgctgccgctgacccacacgaatgctacgctaaggtctttgacgaatttaaaccgctggtggaggagcctcaaaacctcatcaaacagaattgtgaactcttcgaacagctaggagagtacaagttccaaaacgctctactggtgagatatacaaaaaaggtgccgcaagtctcgacaccaacattagtcgaagtttcgcggaaccttggcaaagttggttccaaatgttgcaagcatcctgaagcaaaaaggatgccatgcgcagaagactatttatcagttgtacttaaccaactttgtgtcttgcacgaaaaaactccggtatccgaccgtgtcacgaaatgttgtacggagagcctggtaaaccgccgaccttgcttcagtgctctcgaggtggatgagacctatgtcccaaaagaatttaatgcagaaacgtttaccttccatgcagatatctgtaccctatctgaaaaagagcgccaaattaagaagcaaacggcgcttgttgagttagttaaacataaacctaaagcaacgaaagagcagttaaaggctgtgatggacgacttcgcggcatttgtcgagaaatgctgcaaggctgatgacaaagagacatgttttgccgaggaaggcaagaaactcgttgccgcttctcaggcagccctagggtta

SEQ ID NO: 2 - protein, 585 aa, synthetic construct [sequence omitted]

SEQ ID NO: 3 - DNA, 11037 bp, synthetic construct
  source 1..11037: mol_type = other DNA
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcgccataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatccaaacgatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagcttacgtagaattcaaaagagatgcgcacaaatcagaggtggcgcataggttcaaggaccttggagaagaaaacttcaaggctctagtattgatagcatttgcacaatacttacaacagtgtccatttgaagaccacgtaaaactcgttaatgaggtcactgagttcgccaagacctgcgtggccgatgaatcggcggagaattgtgataaaagtcttcacactcttttcggcgacaagctatgcactgtagcgacattgcgagaaacctacggtgagatggcggattgctgtgccaagcaggagcctgagcgcaatgagtgttttttgcagcacaaagatgacaaccccaatcttcccaggttggtgcggcccgaagttgacgttatgtgcacggcgtttcatgacaacgaagagacttttctgaagaagtatctatatgaaatagcacggagacacccatatttttacgcccctgagctgctcttttttgctaagaggtataaggcggcttttacagaatgttgtcaggcagcggataaggccgcgtgcctcttacccaagttggacgaacttcgagatgaagggaaggcgtctagtgccaagcaacgtctcaagtgcgcatcactccagaagttcggagagagagccttcaaagcttgggcggtggcccgactatcccagcgctttccgaaagctgagttcgcagaggtatcgaagctcgtaaccgaccttacgaaagtacatactgaatgttgccatggtgacttgctggagtgcgcggatgacagagctgatctagcgaaatacatatgtgaaaaccaagatagcattagtagcaagctgaaagaatgctgcgaaaaacccttgttggagaagtctcattgcattgctgaagtagaaaatgacgagatgccggcagaccttccctccttagccgcggatttcgtcgagagcaaggacgtttgtaaaaattacgcagaagccaaggatgtgttcctagggatgtttctgtacgagtatgcccgtcgtcacccggattattcagtcgtcctgctgctccggttagccaaaacatacgagactaccttagaaaaatgctgtgctgccgctgacccacacgaatgctacgctaaggtctttgacgaatttaaaccgctggtggaggagcctcaaaacctcatcaaacagaattgtgaactcttcgaacagctaggagagtacaagttccaaaacgctctactggtgagatatacaaaaaaggtgccgcaagtctcgacaccaacattagtcgaagtttcgcggaaccttggcaaagttggttccaaatgttgcaagcatcctgaagcaaaaaggatgccatgcgcagaagactatttatcagttgtacttaaccaactttgtgtcttgcacgaaaaaactccggtatccgaccgtgtcacgaaatgttgtacggagagcctggtaaaccgccgaccttgcttcagtgctctcgaggtggatgagacctatgtcccaaaagaatttaatgcagaaacgtttaccttccatgcagatatctgtaccctatctgaaaaagagcgccaaattaagaagcaaacggcgcttgttgagttagttaaacataaacctaaagcaacgaaagagcagttaaaggctgtgatggacgacttcgcggcatttgtcgagaaatgctgcaaggctgatgacaaagagacatgttttgccgaggaaggcaagaaactcgttgccgcttctcaggcagccctagggttataataggcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaagttcgtttgtgcaagcttatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctatcgaatctaaatgtaagttaaaatctctaaataattaaataagtcccagtttctccatacgaaccttaacagcattgcggtgagcatctagaccttcaacagcagccagatccatcactgcttggccaatatgtttcagtccctcaggagttacgtcttgtgaagtgatgaacttctggaaggttgcagtgttaactccgctgtattgacgggcatatccgtacgttggcaaagtgtggttggtaccggaggagtaatctccacaactctctggagagtaggcaccaacaaacacagatccagcgtgttgtacttgatcaacataagaagaagcattctcgatttgcaggatcaagtgttcaggagcgtactgattggacatttccaaagcctgctcgtaggttgcaaccgatagggttgtagagtgtgcaatacacttgcgtacaatttcaacccttggcaactgcacagcttggttgtgaacagcatcttcaattctggcaagctccttgtctgtcatatcgacagccaacagaatcacctgggaatcaataccatgttcagcttgagacagaaggtctgaggcaacgaaatctggatcagcgtatttatcagcaataactagaacttcagaaggcccagcaggcatgtcaatactacacagggctgatgtgtcattttgaaccatcatcttggcagcagtaacgaactggtttcctggaccaaatattttgtcacacttaggaacagtttctgttccgtaagccatagcagctactgcctgggcgcctcctgctagcacgatacacttagcaccaaccttgtgggcaacgtagatgacttctggggtaagggtaccatccttcttaggtggagatgcaaaaacaatttctttgcaaccagcaactttggcaggaacacccagcatcagggaagtggaaggcagaattgcggttccaccaggaatatagaggccaactttctcaataggtcttgcaaaacgagagcagactacaccagggcaagtctcaacttgcaacgtctccgttagttgagcttcatggaatttcctgacgttatctatagagagatcaatggctctcttaacgttatctggcaattgcataagttcctctgggaaaggagcttctaacacaggtgtcttcaaagcgactccatcaaacttggcagttagttctaaaagggctttgtcaccattttgacgaacattgtcgacaattggtttgactaattccataatctgttccgttttctggataggacgacgaagggcatcttcaatttcttgtgaggaggccttagaaacgtcaattttgcacaattcaatacgaccttcagaagggacttctttaggtttggattcttctttaggttgttccttggtgtatcctggcttggcatctcctttccttctagtgacctttagggacttcatatccaggtttctctccacctcgtccaacgtcacaccgtacttggcacatctaactaatgcaaaataaaataagtcagcacattcccaggctatatcttccttggatttagcttctgcaagttcatcagcttcctccctaattttagcgttcaacaaaacttcgtcgtcaaataaccgtttggtataagaaccttctggagcattgctcttacgatcccacaaggtggcttccatggctctaagaccctttgattggccaaaacaggaagtgcgttccaagtgacagaaaccaacacctgtttgttcaaccacaaatttcaagcagtctccatcacaatccaattcgatacccagcaacttttgagttgctccagatgtagcacctttataccacaaaccgtgacgacgagattggtagactccagtttgtgtccttatagcctccggaatagactttttggacgagtacaccaggcccaacgagtaattagaagagtcagccaccaaagtagtgaatagaccatcggggcggtcagtagtcaaagacgccaacaaaatttcactgacagggaactttttgacatcttcagaaagttcgtattcagtagtcaattgccgagcatcaataatggggattataccagaagcaacagtggaagtcacatctaccaactttgcggtctcagaaaaagcataaacagttctactaccgccattagtgaaacttttcaaatcgcccagtggagaagaaaaaggcacagcgatactagcattagcgggcaaggatgcaactttatcaaccagggtcctatagataaccctagcgcctgggatcatcctttggacaactctttctgccaaatctaggtccaaaatcacttcattgataccattattgtacaacttgagcaagttgtcgatcagctcctcaaattggtcctctgtaacggatgactcaacttgcacattaacttgaagctcagtcgattgagtgaacttgatcaggttgtgcagctggtcagcagcatagggaaacacggcttttcctaccaaactcaaggaattatcaaactctgcaacacttgcgtatgcaggtagcaagggaaatgtcatacttgaagtcggacagtgagtgtagtcttgagaaattctgaagccgtatttttattatcagtgagtcagtcatcaggagatcctctacgccggacgcatcgtggccgacctgcagggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccaaggcgataaatacccaaatctaaaactcttttaaaacgttaaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatctctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaattctcatgtttgacagcttatcatcgataagctgactcatgttggtattgtgaaatagacgcagatcgggaacactgaaaaataacagttattattcg

SEQ ID NO: 4 - DNA, 12 bp, synthetic construct
  source 1..12: mol_type = other DNA
gaattcaaaaga

SEQ ID NO: 5 - DNA, 14 bp, synthetic construct
  source 1..14: mol_type = other DNA
taataggcggccgc

SEQ ID NO: 6 - DNA, 742 bp, synthetic construct
  source 1..742: mol_type = other DNA
ttattgtttataaatactactattgccagcattgctgctaaagaagaaggggtatctctcgagaaaagagaggctgaagcttacgtagaattcaaaagagatgcgcacaaatcagaggtggcgcataggttcaaggaccttggagaagaaaacttcaaggctctagtattgatagcatttgcacaatacttacaacagtgtccatttgaagaccacgtaaaactcgttaatgaggtcactgagttcgccaagacctgcgtggccgatgaatcggcggagaattgtgataaaagtcttcacactcttttcggcgacaagctatgcactgtagcgacattgcgagaaacctacggtgagatggcggattgctgtgccaagcaggagcctgagcgcaatgagtgttttttgcagcacaaagatgacaaccccaatcttcccaggttggtgcggcccgaagttgacgttatgtgcacggcgtttcatgacaacgaagagacttttctgaagaagtatctatatgaaatagcacggagacacccatatttttacgcccctgagctgctcttttttgctaagaggtataaggcggcttttacagaatgttgtcaggcagcggataaggccgcgtgcctcttacccaagttggacgaacttcgagatgaagggaaggcgtctagtgccaagcaacgtctcaagtgcgcatcactccagaagttcggagagagagccttcaaagcttggg

SEQ ID NO: 7 - DNA, 760 bp, synthetic construct
  source 1..760: mol_type = other DNA
tcaagtgcgcatcactccagaagttcggagagagagccttcaaagcttgggcggtggcccgactatcccagcgctttccgaaagctgagttcgcagaggtatcgaagctcgtaaccgaccttacgaaagtacatactgaatgttgccatggtgacttgctggagtgcgcggatgacagagctgatctagcgaaatacatatgtgaaaaccaagatagcattagtagcaagctgaaagaatgctgcgaaaaacccttgttggagaagtctcattgcattgctgaagtagaaaatgacgagatgccggcagaccttccctccttagccgcggatttcgtcgagagcaaggacgtttgtaaaaattacgcagaagccaaggatgtgttcctagggatgtttctgtacgagtatgcccgtcgtcacccggattattcagtcgtcctgctgctccggttagccaaaacatacgagactaccttagaaaaatgctgtgctgccgctgacccacacgaatgctacgctaaggtctttgacgaatttaaaccgctggtggaggagcctcaaaacctcatcaaacagaattgtgaactcttcgaacagctaggagagtacaagttccaaaacgctctactggtgagatatacaaaaaaggtgccgcaagtctcgacaccaacattagtcgaagtttcgcggaaccttggcaaagttggttccaaatgttgcaagcatcctgaagcaaaaaggatgccatgcgcagaaga

SEQ ID NO: 8 - DNA, 766 bp, synthetic construct
  source 1..766: mol_type = other DNA
atatacaaaaaaggtgccgcaagtctcgacaccaacattagtcgaagtttcgcggaaccttggcaaagttggttccaaatgttgcaagcatcctgaagcaaaaaggatgccatgcgcagaagactatttatcagttgtacttaaccaactttgtgtcttgcacgaaaaaactccggtatccgaccgtgtcacgaaatgttgtacggagagcctggtaaaccgccgaccttgcttcagtgctctcgaggtggatgagacctatgtcccaaaagaatttaatgcagaaacgtttaccttccatgcagatatctgtaccctatctgaaaaagagcgccaaattaagaagcaaacggcgcttgttgagttagttaaacataaacctaaagcaacgaaagagcagttaaaggctgtgatggacgacttcgcggcatttgtcgagaaatgctgcaaggctgatgacaaagagacatgttttgccgaggaaggcaagaaactcgttgccgcttctcaggcagccctagggttataataggcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagc

SEQ ID NO: 9 - DNA, 10 bp, synthetic construct
  source 1..10: mol_type = other DNA
ccaaacgatg

(19) United States
(12) Patent Application Publication
(10) Pub. No.: US 2026/0209784 A1
(43) Pub. Date: Jul. 23, 2026

(54) METHOD FOR CONSTRUCTING HOST STRAIN CAPABLE OF HIGHLY EXPRESSING HUMAN SERUM ALBUMIN

(71) Applicant: HEBEI HUAKAI HUIHE BIOMEDICAL CO., LTD., Baoding (CN)

(72) Inventors: Zhimin LIU, Baoding (CN); Yueqiang ZHAO, Baoding (CN)

(73) Assignee: HEBEI HUAKAI HUIHE BIOMEDICAL CO., LTD., Baoding (CN)

(21) Appl. No.: 19/634,139

(22) Filed: Mar. 31, 2026

Related U.S. Application Data
(63) Continuation-in-part of application No. PCT/CN2025/114082, filed on Aug. 12, 2025.

(30) Foreign Application Priority Data
Aug. 13, 2024 (CN) 202411108150.4

(51) Int. Cl. C12N 15/81; C07K 14/765; C12N 1/16; C12N 9/16; C12N 15/64; C12R 1/84

(52) U.S. Cl. CPC C12N 15/815; C07K 14/765; C12N 1/16; C12N 9/16; C12N 15/64; C12Y 301/21004; C12N 2830/34; C12R 2001/84

(57) ABSTRACT

A method for constructing a host strain capable of highly expressing human serum albumin (HSA) includes: designing an optimized gene encoding the HSA; constructing a recombinant expression vector; and transforming the recombinant expression vector into a Pichia pastoris host strain through electroporation to produce the host strain. Through the molecular-level optimization design for the full sequence of the target gene, a target gene fragment most suitable for the expression in Pichia pastoris is acquired. At the cellular level, the internationally latest Pichia pastoris expression system host strain CBS7435 is introduced to express the gene for HSA. Pichia pastoris transformants carrying the exogenous gene with a high copy number are selected with the geneticin (G418) resistance gene to construct an engineered strain for expressing recombinant HSA at a high level. The method can be used to prepare HSA with a purity of 99% or more.

### CROSS REFERENCE TO THE RELATED APPLICATIONS

This application is a continuation-in-part application of International Application No. PCT/CN2025/114082, filed on Aug. 12, 2025, which is based upon and claims priority to Chinese Patent Application No. 202411108150.4, filed on Aug. 13, 2024, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named ZDSH0503P_SequenceListing.xml, created on 02/10/2026, and is 24,388 bytes in size.

### TECHNICAL FIELD

The present patent disclosure belongs to the biotechnology field of preparing human serum albumin (HSA) based on genetic engineering, and specifically relates to a method for constructing a host strain capable of highly expressing HSA.

### BACKGROUND

The development of molecular biotechnology has provided numerous approaches and means for the large-scale commercial production of exogenous proteins with bioreactors. So far, various expression systems for exogenous proteins have been developed, including Escherichia coli (E. coli), yeast, insect, and mammalian cell systems. The Pichia pastoris gene expression system, after nearly three decades of development, has become one of the most important hosts for expressing exogenous proteins, such as the host strain GS115 (Cregg, et al. (2009). Methods Enzymol. 463, 169-189; U.S. Pat. No. 4,879,231, Phillips Petroleum, 1989). The Pichia pastoris gene expression system offers advantages such as easy high-density fermentation, stable integration of target genes into the host genome, effective secretion and appropriate glycosylation of expressed products, and cost-effective media. Through the highly-efficient and regulatable AOX1 promoter, the Pichia pastoris gene expression system has achieved the high-level expression of thousands of exogenous proteins, including hepatitis B surface antigen (HBsAg), tumor necrosis factor (TNF), epidermal growth factor (EGF), tetanus toxin fragment C, and genetically engineered antibodies. It has been proved that this gene expression system is efficient, practical, and simple, is predominantly characterized by the enhancement of expression levels and the retention of biological activities of products, and is highly suitable for pilot-scale amplification and large-scale industrial production.

The expression of exogenous genes in Pichia pastoris typically includes the following steps: (1) An exogenous gene is inserted into a Pichia pastoris expression vector to construct a recombinant expression vector. (2) The recombinant expression vector is digested and treated with endonucleases to obtain a linearized recombinant plasmid and then transformed into a Pichia pastoris strain. (3) A transformant solution is coated on MD plates for the first round of screening of positive recombinants. (4) The second round of screening of positive recombinants is conducted with YPD plates including geneticin G418 at different concentrations. (5) The integration of the exogenous gene into the yeast genome is further identified. (6) An expression level of the exogenous gene is determined through small-scale induced expression. (7) Large-scale fermentation production is conducted with a bioreactor, and a recombinant protein is extracted from a fermentation broth. To improve the expression level of an exogenous gene in Pichia pastoris, it is usually necessary to select Pichia pastoris transformants carrying the exogenous gene with a high copy number. As a result, the gene dosage in the host strain increases. The selection of high-copy-number transformants is achieved by progressively elevating the resistance to G418, which is inherently highly tedious and random. The further investigation shows that the relationship between high-copy-number Pichia pastoris transformants and highly-expressed exogenous proteins remains variable. Because the optimal copy number varies depending on the target gene, there is no linear relationship between high-copy-number Pichia pastoris transformants and highly-expressed exogenous proteins (see the Pichia pastoris Laboratory Manual). In the series of recombinant expression vectors such as pPIC9K and pPIC3.5K commonly used for Pichia pastoris, a G418 resistance gene has been incorporated. After these recombinant expression vectors are transformed into Pichia pastoris, both an exogenous gene and the resistance gene are integrated into a chromosome of Pichia pastoris through homologous recombination. By increasing the concentration of G418 for resistance screening, transformants carrying high-copy insertions of the exogenous gene, which represent a minority in a transformant population, can be selected.

To select positive clones carrying the exogenous gene with a high copy number, the screening needs to be conducted with G418 gradient plates with different concentrations. In the preliminary experiment, G418 concentrations are set to 0 mg/mL, 0.25 mg/mL, 0.5 mg/mL, 0.75 mg/mL, 1.0 mg/mL, 1.5 mg/mL, 1.75 mg/mL, 2.0 mg/mL, 3.0 mg/mL, and 4.0 mg/mL. According to the protocol (Pichia Expression Kit: A Manual of Methods for Expression of Recombinant Proteins in Pichia pastoris, Catalog No. K1710-01) recommended by Invitrogen, the following screening methods are described: 1. G418 gradient plates with different concentrations are prepared, and positive clones are then transferred from an HIS4 auxotrophic plate one by one to these G418 gradient plates through replica plating to achieve the screening of high-copy-number clones. This method is characterized by immense workload (three consecutive subcultures are required before replica plating to ensure equivalent densities for individual transformants), high screening difficulty, prolonged cycle, and relatively-limited number of screened transformants, making it unsuitable for high-throughput screening. 2. G418 gradient plates with different concentrations are prepared, and all positive transformants growing on an HIS4 auxotrophic plate can be washed off with sterile water or a liquid medium, then diluted to an appropriate concentration, and then coated at a specified density on the G418 gradient plates with different concentrations. This method involves relatively simple operations and enables the screening of a larger number of transformants. However, for different target genes or different Pichia pastoris host strains, a preliminary experiment is required to determine the optimal dilution factor for a strain suspension. The above two screening methods are currently the universal screening methods employed by scientific researchers worldwide in the production of exogenous proteins with the Pichia pastoris expression system. However, both of the two screening methods demonstrate drawbacks including high G418 consumption, cumbersome procedures, heavy workload, high time and labor consumption, and poor universality across different Pichia pastoris host strains.

### SUMMARY

### 1. Technical Problem to be Solved by the Present Disclosure

An objective of the present patent disclosure is to address the following problems: The existing technique for preparing HSA through genetic recombination has drawbacks such as low gene expression levels and immature fermentation process control (due to the large scale of fermentation, 10-ton or dozens-of-ton large fermenters are often required, and there is a lack of precedents and tools for process control and monitoring means for large fermenters). The existing purification process for recombinant HSA is immature (since recombinant HSA is a special case, the purification scale and equipment for the target protein are exceptionally large, and the target protein needs to have an extremely-high purity of 99.99% or more). That is, there are problems such as tens of purification steps of the recombinant protein, low purification medium load, low purification efficiency, and low purification recovery rate.

### 2. Technical Solution

In order to achieve the above objective, the present disclosure provides the following technical solutions:

The present disclosure provides a method for constructing a host strain capable of highly expressing HSA, including:
S100, designing an optimized gene encoding the HSA;
S200, constructing a recombinant expression vector shown in SEQ ID NO: 3; and
S300, transforming the recombinant expression vector obtained in the step S200 into a Pichia pastoris host strain through electroporation to produce the host strain.

Preferably, for the optimized gene encoding the HSA in the step S100, a nucleotide sequence of the optimized gene is shown in SEQ ID NO: 1 and an amino acid sequence of the HSA is shown in SEQ ID NO: 2.

Preferably, in the optimized gene encoding the HSA in the step S100, optimal codons preferred by an alcohol oxidase 1 (AOX1) gene derived from Pichia pastoris are adopted, and a proportion of the optimal codons in total codons of the optimized gene is controlled at 90%.

Preferably, three restriction endonuclease sites SalI, HindIII, and XbaI are inserted sequentially in a 5′ to 3′ direction within the optimized gene encoding the HSA in the step S100, such that the optimized gene is divided into four relatively-balanced large fragments.

Preferably, in the optimized gene encoding the HSA in the step S100, consecutive G-C base pairs are reduced and A-T base pairs preferred by the Pichia pastoris are increased, achieving a balanced design of the nucleotide distribution within the optimized gene, such that a GC content in the optimized gene is adjusted to 45% to 50% (See FIG. 1).

Preferably, the optimized gene encoding the HSA in the step S100 further includes an expression reading cassette constructed as follows:
inserting a 5′ restriction endonuclease site BamI into a 5′ regulatory region (promoter region) of the AOX1 gene, ligating a 10-deoxynucleotide oligonucleotide CCAAACGATG as shown in SEQ ID NO: 9 (including a Kozak sequence for eukaryotic genes, which is AXXATG), ligating a yeast α-mating factor leader peptide sequence (including 85 amino acids) derived from Saccharomyces cerevisiae, and inserting a mature HSA gene between multiple cloning sites EcoRI and NotI of a pPIC9K recombinant expression vector; and
inserting a coding sequence AAAAGA for a dibasic amino acid of lysine and arginine (-Lys-Arg-) after an enzyme cleavage site EcoRI at a 5′ terminus of the mature HSA gene, where the specific sequence is as shown in SEQ ID NO: 4; and inserting a double stop codon TAATAG before an enzyme cleavage site NotI at a 3′ terminus of the mature HSA gene, where the specific sequence is as shown in SEQ ID NO: 5.

Preferably, the recombinant expression vector constructed in the step S200 is as follows:
excising the optimized gene encoding the HSA fully chemically synthesized from a cloning vector through EcoRI-NotI double enzyme digestion, cutting a gel band with an expected size off, and recovering a target gene-containing fragment with a DNA recovery kit; and cloning the target gene-containing fragment into a recombinant expression vector undergoing corresponding double enzyme digestion to produce the recombinant expression vector.

Preferably, the step S300 is as follows: linearizing the recombinant expression vector obtained in the step S200 with a restriction endonuclease SacI or BglII, and transforming a linearized recombinant expression vector into the Pichia pastoris host strain through the electroporation to produce the host strain, such as X33, GS115, KM71, SMD1168, CBS7435, NCYC2543, etc.

Preferably, the nucleotide sequence of the recombinant expression vector is shown in SEQ ID NO: 1.

Preferably, the recombinant expression vector is any one of pPIC9, pPIC3, pPICZαABC, pPIC3.5K, pHIL-S1, pHIL-D2, pA0804, pA0815, pGAPZαABC, pPIC6αABC, and pPIC9K.

Preferably, the recombinant expression vector is HSA-pPIC9K.

Preferably, the Pichia pastoris host strains are novel Pichia pastoris host CBS7435, NCYC2543.

Preferably, the host strain is Pichia pastoris HSA-C16, which was deposited at China General Microbiological Culture Collection Center (CGMCC) on Mar. 28, 2024, with an accession number of CGMCC No. 30175.

### 3. Beneficial Effects

Compared with the prior art, the technical solutions provided by the present disclosure have the following beneficial effects:

The present disclosure provides a method for constructing a host strain capable of highly expressing HSA, including the following steps: S100. An optimized gene encoding the HSA is designed. S200. A recombinant expression vector is constructed. S300. The recombinant expression vector obtained in the step S200 is transformed into a Pichia pastoris host strain through electroporation to produce the host strain. Through the molecular-level optimization design for the full sequence of the target gene, a target gene fragment most suitable for the expression in Pichia pastoris is acquired. At the cellular level, the internationally latest Pichia pastoris expression system host strain CBS7435 is introduced to express the gene for HSA. Pichia pastoris transformants carrying the exogenous gene with a high copy number are selected with the G418 resistance gene to construct an engineered strain for expressing recombinant HSA at a high level. The method can be used to prepare HSA with a purity of 99% or more.

### Deposition of Biological Material

The Pichia pastoris HSA-C16 was deposited at the China General Microbiological Culture Collection Center (CGMCC), Institute of Microbiology Chinese Academy of Sciences No. 1 West Beichen Road, Chaoyang District, Beijing, China on Mar. 28, 2024, with an accession number of CGMCC No. 30175.

### BRIEF DESCRIPTION OF THE DRAWINGS

The application file contains at least one drawing executed in color. Copies of this patent or patent application publication with color drawing(s) will be provided by the Office upon request and payment of the necessary fee.

FIG. 1 is a schematic diagram of a computer-simulated alignment curve for the adjustment of a G-C base pair content within a nucleotide sequence shown in SEQ ID NO: 1 for an optimized gene to an optimal level in an embodiment;

FIG. 2 is a map of a Pichia pastoris multi-copy recombinant expression vector pPIC9K in an embodiment;

FIGS. 3A-3B show agarose gel electrophoresis (1% agarose gel) results for the optimized gene in an embodiment;

FIG. 4 is a schematic diagram of a construction process of a recombinant plasmid in an embodiment;

FIG. 5 is a first analysis profile for a nucleotide sequence of the optimized gene in an embodiment, where sequencing results of the nucleotide acid sequence were shown in SEQ ID NO: 6;

FIG. 6 is a second analysis profile for the nucleotide sequence of the optimized gene in an embodiment, where sequencing results of the nucleotide acid sequence were shown in SEQ ID NO: 7;

FIG. 7 is a third analysis profile for the nucleotide sequence of the optimized gene in an embodiment, where sequencing results of the nucleotide acid sequence were shown in SEQ ID NO: 8;

FIG. 8 is a schematic diagram illustrating cell state changes during the electroporation-mediated transformation of the recombinant plasmid into a Pichia pastoris host strain in an embodiment;

FIG. 9 is a schematic diagram illustrating cell state changes during the electroporation-mediated transformation of the recombinant plasmid into a Pichia pastoris host strain GS115 in an embodiment;

FIG. 10 is a schematic diagram illustrating cell state changes during the electroporation-mediated transformation of the recombinant plasmid into a Pichia pastoris host strain CBS7435 in an embodiment;

FIG. 11 shows a first set of results from a small-scale induction experiment for a Pichia pastoris GS115 engineered strain prepared in an embodiment;

FIG. 12 shows a second set of results from a small-scale induction experiment for the Pichia pastoris GS115 engineered strain prepared in the embodiment;

FIG. 13 shows a first set of results from a small-scale induction experiment for a Pichia pastoris CBS7435 engineered strain prepared in an embodiment;

FIG. 14 shows a second set of results from a small-scale induction experiment for the Pichia pastoris CBS7435 engineered strain prepared in the embodiment;

FIG. 15 is a first schematic diagram of protein electrophoresis results at different time points during a 50 L fermenter fermentation test of a Pichia pastoris engineered strain GS115 (G15) prepared in an embodiment;

FIG. 16 is a second schematic diagram of protein electrophoresis results at different time points during a 50 L fermenter fermentation test of the Pichia pastoris engineered strain CBS7435 (C7) prepared in the embodiment;

FIG. 17 is a third schematic diagram of protein electrophoresis results at different time points during a 500 L fermenter fermentation test of the Pichia pastoris engineered strain CBS7435 (C16) prepared in the embodiment;

FIG. 18 is a third schematic diagram of protein electrophoresis results at different time points during a 2000 L fermenter fermentation test of the Pichia pastoris engineered strain CBS7435 (C16) prepared in the embodiment;

FIG. 19 shows a standard curve for quantitative determination of a protein concentration with bovine serum albumin as a standard in an embodiment;

FIG. 20 is a schematic diagram of separation and purification results for a supernatant sample from a 50 L fermenter fermentation of an engineered strain prepared in an embodiment;

FIG. 21 shows purity identification data of a human serum albumin sample purified by HPLC analysis in an embodiment;

FIG. 22 is a comparative schematic diagram of non-reduced molecular-weight total ion chromatogram (TIC) analysis between the human serum albumin sample purified in the embodiment and an rHSA national standard;

FIG. 23 is a comparative schematic diagram of reduced molecular-weight TIC analysis between the human serum albumin sample purified in the embodiment and the rHSA national standard; and

FIG. 24 is a comparative schematic diagram of reduced molecular-weight mass spectrometry analysis between the human serum albumin sample purified in the embodiment and the rHSA national standard.

### BRIEF DESCRIPTION OF THE SEQUENCE LISTING

SEQ ID NO: 1 is an artificial DNA sequence of the optimized target gene sequence (i.e. human serum albumin cDNA Length: 1-1755 bp) synthesized in the embodiment;

SEQ ID NO: 2 is an amino acid sequence (Sequence Length: 585 aa) corresponding to the optimized target gene sequence of SEQ ID NO: 1;

SEQ ID NO: 3 is a full sequence analytical results of a constructed recombinant plasmid of HSA-pPIC9K in an embodiment;

SEQ ID NO: 4 is an additional 5′ sequence before the optimized target gene sequence inserted two codons in an embodiment;

SEQ ID NO: 5 is an additional 3′ sequence after the optimized target gene sequence inserted two stop codons in an embodiment;

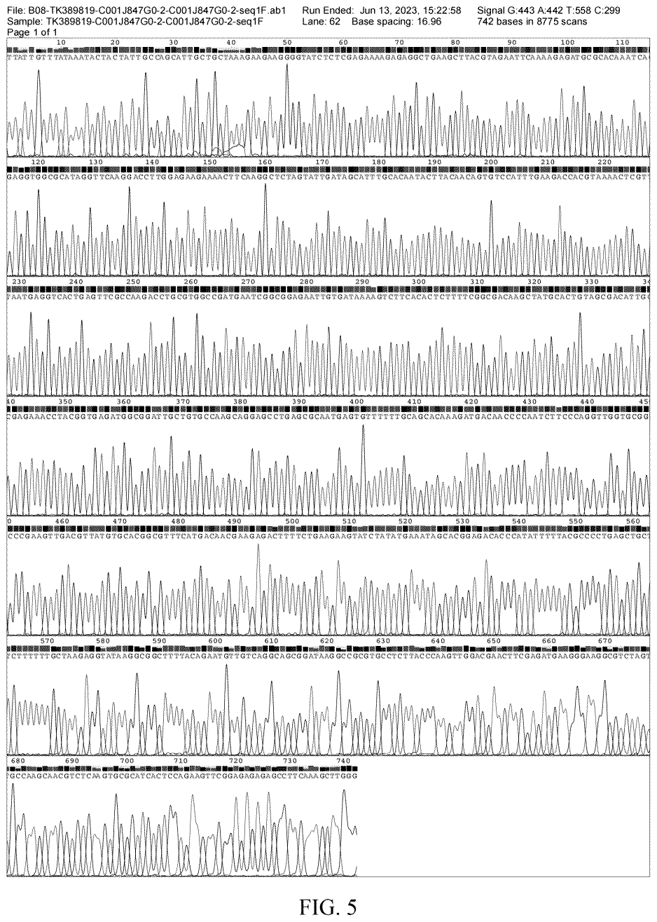

SEQ ID NO: 6 is a sequence alignment analysis of sequencing result (SEQ contig) corresponding to FIG. 5 which is a first analysis profile for a nucleotide sequence of the optimized gene in an embodiment;

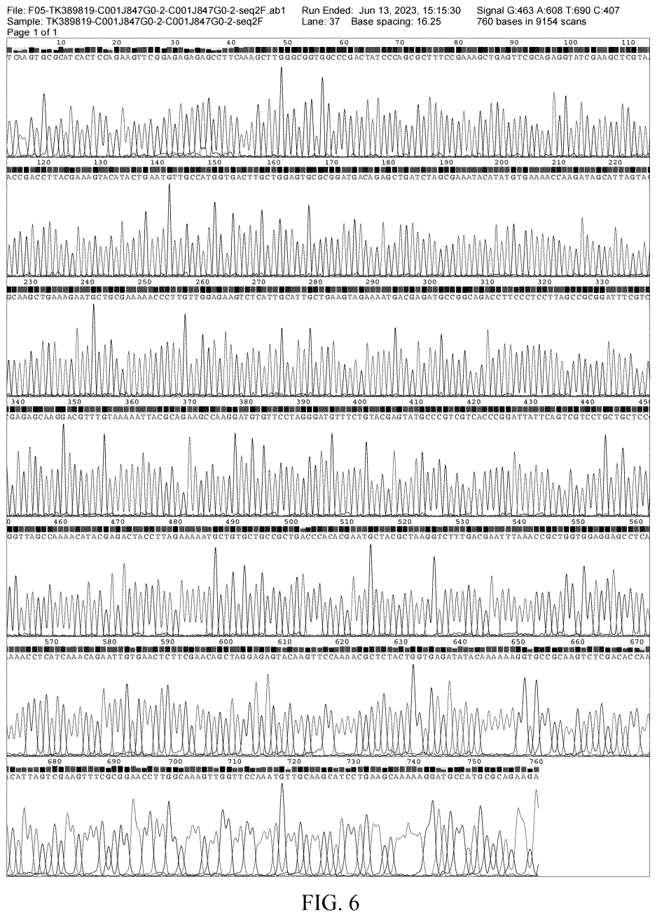

SEQ ID NO: 7 is a sequence alignment analysis of sequencing result (SEQ contig) corresponding to FIG. 6 which is a second analysis profile for a nucleotide sequence of the optimized gene in an embodiment;

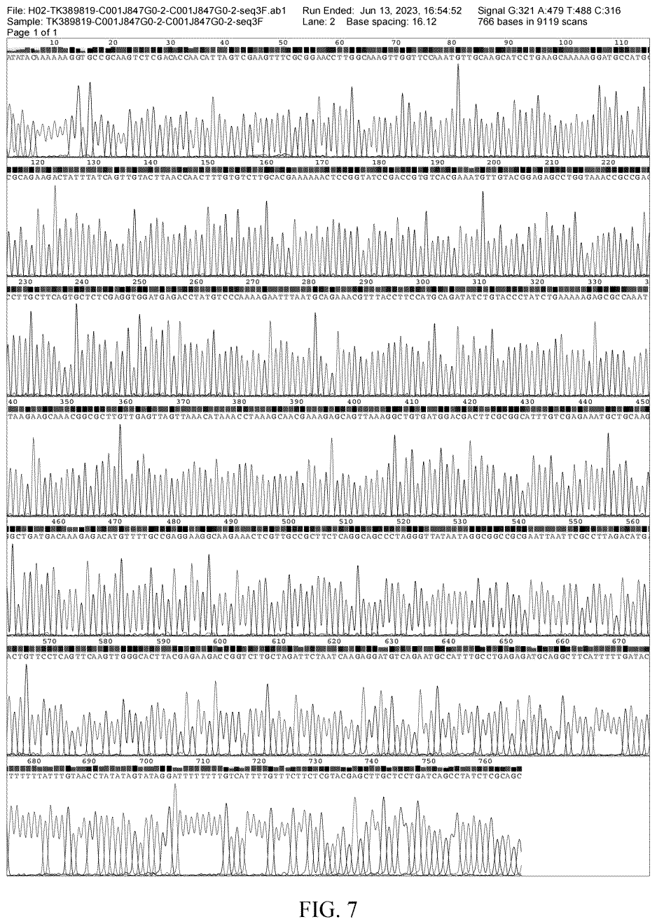

SEQ ID NO: 8 is a sequence alignment analysis of sequencing result (SEQ contig) corresponding to FIG. 7 which is a third analysis profile for a nucleotide sequence of the optimized gene in an embodiment.

### DETAILED DESCRIPTION OF THE EMBODIMENTS

To enable those skilled in the art to well understand the solutions of the present application, the technical solutions in the embodiments of the present application will be clearly and completely described below in conjunction with the accompanying drawings in the embodiments of the present application. Obviously, the described embodiments are only some rather than all of the embodiments of the present application. All other embodiments obtained by those of ordinary skill in the art based on the embodiments of the present application without creative efforts should fall within the protection scope of the present application.

It should be noted that the terms “first”, “second”, etc. In the description and claims of the present application and in the above accompanying drawings are intended to distinguish between similar objects, but do not necessarily indicate a specific order or sequence. It should be understood that data used in such contexts may be interchanged under appropriate circumstances to enable the embodiments of the present application described herein. Moreover, the terms “include”, “has”, and any variants thereof refer to non-exclusive inclusion, for example, a process, method, system, product, or device that includes a series of steps or units is not necessarily limited to those steps or units which are clearly listed, but may include other steps or units which are not expressly listed or inherent to such a process, method, system, product, or device.

In the present application, orientations or positional relationships indicated by terms such as “above”, “below”, “left”, “right”, “front”, “rear”, “top”, “bottom”, “inside”, “outside”, “vertical”, “horizontal”, “transverse”, and “longitudinal” are all based on the orientations or positional relationships illustrated in the accompanying drawings. These terms are mainly intended to well describe the present application and embodiments thereof, rather than to define that devices, elements, or components indicated by these terms must have the specific orientations or must be constructed and operated in the specific orientations.

Besides, some of the terms mentioned above may be used to indicate other meanings in addition to the orientations or positional relationships. For example, the term “upper” may also be used to indicate an attachment relationship or a connection relationship in some cases. Those of ordinary skill in the art may understand specific meanings of these terms in the present application based on a specific situation.

In addition, the terms “mounted”, “arranged”, “provided”, and “connected” should be interpreted in a broad sense. For example, “connection” may be a fixed connection, a removable connection, or integration; may be a mechanical connection or an electrical connection; may be a direct connection or an indirect connection implemented through an intermediate medium; or may be intercommunication between two devices, elements, or components. Those of ordinary skill in the art may understand specific meanings of the above terms in the present application based on a specific situation.

It should be noted that the embodiments in the present application and features in the embodiments may be combined with each other in a non-conflicting situation. The present application will be described in detail below with reference to the accompanying drawings and embodiments.

### Example 1

### The Conventional Experimental Strains, Materials, Instruments and Equipment, and Experimental Methods of the Present Invention

In this example, a method for constructing a host strain capable of highly expressing HSA is provided, including:
S100: An optimized gene encoding the HSA was designed.
S200: A recombinant expression vector was constructed.
S300: The recombinant expression vector obtained in the step S200 was transformed into a Pichia pastoris host strain through electroporation to produce the host strain.

For the optimized gene encoding the HSA in the step S100, a nucleotide sequence of the optimized gene is shown in SEQ ID NO: 1 and an amino acid sequence of the HSA is shown in SEQ ID NO: 2.

In the optimized gene encoding the HSA in the step S100, optimal codons preferred by an AOX1 gene derived from Pichia pastoris are adopted. A proportion of the optimal codons in total codons of the optimized gene is controlled at 90%.

Three restriction endonuclease sites SalI, HindIII, and XbaI are inserted sequentially in a 5′ to 3′ direction within the optimized gene encoding the HSA in the step S100, such that the optimized gene is divided into four relatively-balanced large fragments.

In the optimized gene encoding the HSA in the step S100, consecutive G-C base pairs are reduced and A-T base pairs preferred by the Pichia pastoris are increased, such that a GC content in the optimized gene is adjusted to 45% to 50%.

Preferably, the optimized gene encoding the HSA in the step S100 further includes an expression reading cassette constructed as follows:

A 5′ restriction endonuclease site BamHI is inserted into a 5′ regulatory region (promoter region) of the AOX1 gene. A 10-deoxynucleotide oligonucleotide CCAAACGATG as shown in SEQ ID NO: 9 (including a Kozak sequence for eukaryotic genes, which is AXXATG) is ligated. A yeast α-mating factor leader peptide sequence (including 85 amino acids) derived from Saccharomyces cerevisiae is then ligated. A mature HSA gene is inserted between multiple cloning sites EcoRI and NotI of a pPIC9K recombinant expression vector.

A coding sequence AAAAGA for a dibasic amino acid of lysine and arginine (-Lys-Arg-) is inserted after an enzyme cleavage site EcoRI at a 5′ terminus of the mature HSA gene, where the specific sequence is as shown in SEQ ID NO: 4. A double stop codon TAATAG is inserted before an enzyme cleavage site NotI at a 3′ terminus of the mature HSA gene, where the specific sequence is as shown in SEQ ID NO: 5.

The construction of a recombinant expression vector in step S200 is as follows:

The optimized gene encoding the HSA fully chemically synthesized is excised from a cloning vector through EcoRI-NotI double enzyme digestion, a gel band with an expected size is cut off, and a target gene-containing fragment is recovered with a DNA recovery kit. The target gene-containing fragment is cloned into a recombinant expression vector undergoing corresponding double enzyme digestion to produce the recombinant expression vector.

The step S300 is as follows: The recombinant expression vector obtained in the step S200 is linearized with a restriction endonuclease SacI or BglII, and a linearized recombinant expression vector is then transformed into the Pichia pastoris host strain through the electroporation to produce the host strain.

A nucleotide sequence of the recombinant expression vector is shown in SEQ ID NO: 1.

The recombinant expression vector is any one of pPIC9, pPIC3, pPICZαABC, pPIC3.5K, pHIL-S1, pHIL-D2, pA0804, pA0815, pGAPZαABC, pPIC6αABC, and pPIC9K.

The recombinant expression vector is HSA-pPIC9K.

The Pichia pastoris host strain is Pichia pastoris CBS7435.

Procedures and experimental data related to the construction of a host strain capable of highly expressing HSA in this example are as follows:

### Materials and Devices:

### 1. Strains

1) Pichia pastoris host strain (GS115 his4 (Mut⁺ his⁻)NRRL Y-15851).

2) Pichia pastoris host strain CBS7435 [the host strain nowadays reclassified as Komagataella phaffi (formerly named Pichia pastoris), named NRRL Y-11430 ]; Komagataella phaffi species NCYC2543.

3) E. coli host strains:
E. coli JM10F′(endA1, recA1, gyrA96,thi, sdR17(rₖ⁻,mₖ⁺),relA1,supE44,Δ(lac-proAB), [F′ traD36, proAB, lacI^{q}ZΔM15]; and
E. coli HB101 (supE44 hsd S20(rB-mB-)recA:ara-14 proA₂ lac Y:galK₂rpsL20 xyl-5 mtl-1).

The above-mentioned E. coli host strains were used for the cloning of the target gene and the construction and preparation of plasmids.

### 2. Main Reagents

1) DNA restriction endonucleases, T₄ DNA ligase, and polymerases were purchased from GIBCO-BRL, Pharmacia, Bio-Labs, and Cusabio, respectively.

2) Casamino acid (MERK, Germany).

3) Bacto-yeast extract (Difco, USA).

4) Polymerase chain reaction (PCR) amplification kit (Pharmacia, Sweden).

5) DNA sequencing kit (USB, USA).

6) Acrylamide (Acr), N,N-dimethylbisacrylamide (Bis), sodium dodecyl sulfate (SDS), guanidine hydrochloride, urea, and tetramethylethylenediamine (TEMED) (Sigma, UK).

7) Isopropylthio-β-galactoside (IPTG), X-gal, dithiothreitol (DTT), and Agarose (Sigma, UK).

8) YNB, Biotin, and Agar (Difco, USA).

9) Sorbitol, glucose, L-histidine, L-lysine, L-methionine, L-Leucine, L-isoleucine, and L-glutamic acid (Sigma, USA).

10) Glycerol and methanol (Shanghai Chemical Reagent Factory).

11) Enzyme reaction solutions:

Restriction endonuclease high-salt buffer: 10 mM Tris-HCl (pH 7.5), 100 mM NaCl, 10 mM MgCl₂.

Restriction endonuclease medium-salt buffer: 50 mM Tris-HCl (pH 7.5), 50 mM NaCl, 10 mM MgCl₂.

Restriction endonuclease low-salt buffer: 10 mM Tris-HCl (pH 8.0), 10 mM MgCl₂.

T₄ DNA ligase buffer: 50 mM Tris-HCl (pH 8.0), 10 mM MgCl₂, 10 mM DTT, 1 mM ATP.

12) Sodium dodecyl sulfate-polyacrylamide gel electrophoresis (SDS-PAGE) reagents:

Electrophoresis buffer: 192 mM glycine, 25 mM Tris-HCl, and 0.1% SDS, pH 8.3.

Stacking gel buffer: 125 mM Tris-HCl and 0.1% SDS, pH 6.8.

Separating gel buffer: 375 mM Tris-HCl, 0.1% SDS, pH 8.8.

Sample buffer (1×): 50 mM Tris-HCl (pH 6.8), 1% SDS, 10% glycerol, 2.5% mercaptoethanol, and 0.05% bromophenol blue.

30% Acr: 29% Acr, 1% N,N-dimethylbisacrylamide.

Coomassie Brilliant Blue staining solution: 0.25% (W/V) Coomassie Brilliant Blue G-250 and 5% HAc.

45% ethanol destaining solution: 7.5% HAc and 10% ethanol.

13) Common buffers:

TE buffer: 10 mM Tris-HCl (pH 8.0) and 1 mM EDTA.

STE buffer: 10 mM Tris-HCl (pH 8.0), 1 mM EDTA, and 20 mM NaCl.

Phosphate-buffered saline (PBS): 10 mM NaH, P0q-Na, HPO₃ (pH 7.0), and 150 mM NaCl.

10×TBS buffer: per liter: 108 g of Tris base, 55 g of boric acid, and 40 mL of EDTA (0.5 M), pH 8.0.

50×TAE buffer: per liter: 242 g of Tris base, 57.1 mL of glacial acetic acid, and 100 mL of EDTA (0.5 M), pH 8.0.

Saturated phenol: Commercially-available phenol was redistilled, saturated with TE buffer, dispensed, and stored at −20° C.

Phenol:chloroform:isoamyl alcohol (V/V): 1:1:0.8.

Chloroform:isoamyl alcohol (V/V): 24:1.

### 3. Main Instruments and Devices

1) ABI381A DNA automatic synthesizer, Applied Biosystems (ABI), USA.

2) 5 L RIBE-5 automatic fermenter, Shanghai Guoqiang Biochemical Engineering Equipment Co., Ltd.

3) 15 L Biocenter 15F automatic fermenter, Shanghai Guoqiang Biochemical Engineering Equipment Co., Ltd. (Software system: Fermentation Star).

4) 50 L Biocenter 50F automatic fermenter, Shanghai Guoqiang Biochemical Engineering Equipment Co., Ltd. (Software system: Fermentation Star).

5) 100 L Biocenter 100F automatic fermenter, Shanghai Guoqiang Biochemical Engineering Equipment Co., Ltd. (Software system: Fermentation Star).

6) 500 L automatic BIOSTAT® Bplus bioreactor, B. Braun, Germany.

7) 2000 L automatic BLBIO-2000SJ bioreactor, Shanghai Bailun Biotechnology Co., Ltd.

8) SDL-100 protein purification system, Suzhou SePure Instruments Co., Ltd. (medium-pressure chromatography workstation, software SCG).

9) Agilent High-Performance Liquid Chromatograph, 1260 Infinity, Agilent Technologies, USA.

10) External-pressure ultrafiltration membrane module (filtration accuracy: 30 KD), Huzhou MFL Membrane Technology Co., Ltd.

11) External-pressure ultrafiltration membrane module (filtration accuracy: 100 KD), Huzhou MFL Membrane Technology Co., Ltd.

12) Full-wavelength microplate reader, Thermo Fisher 1510, Thermo Fisher Scientific, USA.

13) Electroporation instrument, Micropulser 411BR10654, BIO-RAD, USA.

### 4. Experimental Methods

### (1) Media and Culture Conditions

E. coli liquid medium LB (1% of bacto-tryptone, 0.5% of bacto-yeast extract, and 1% of NaCl). A corresponding solid medium was produced by adding an agar powder at 15 g/L to the liquid medium. E. coli was cultured at 37° C. Pichia pastoris rich medium YPD (1% of yeast extract, 2% of peptone, and 2% of dextrose). Pichia pastoris protoplast regeneration medium RDB [1 M of sorbitol, 1% of dextrose, 1.34% of YNB, 4×10⁵ of biotin, 0.005% of an amino acid mixture (including L-glutamic acid, L-methionine, L-leucine, L-isoleucine, and L-lysine)]. A corresponding solid medium was produced by adding an agar powder at 2% to the liquid medium. Pichia pastoris shake flask media BMGY and BMMY (1% of yeast extract, 2% of peptone, 100 mM of PBS, pH 6.0, 1.34% of YNB, 4×10⁵% of biotin, 1% of glycerol, or 0.5% of methanol). Pichia pastoris was cultured at 30° C.

(2) First High-Density Fermentation Medium and Culture Conditions for Pichia pastoris in a Fermenter:

| A. 10 × Basal Salts: | | |
|---|---|---|
| 1. H₃PO₄, 85% | 42 | ml |
| 2. CaSO₂•2H₂ O | 1.8 | g/L |
| 3. K₂SO₄ | 28.6 | g/L |
| 4. MgSO₄•7H₂ O | 23.4 | g/L |
| 5. KOH | 6.5 | g/L |
| B. 250 × PTMl salts: | | |
| 1. CuSO₄•5H₂ O | 6 | g/L |
| 2. KI | 0.08 | g/L |
| 3. MnSO₄•H₂O | 3 | g/L |
| 4. Na₂MoO₄•2H₂ O | 0.2 | g/L |
| 5. H₃BO₃ | 0.02 | g/L |
| 6. CoCl₂ | 0.5 | g/L |
| 7. ZnCl₂ | 20 | g/L |
| 8. FeSO₄•7H₂O | 65 | g/L |
| 9. Biotin | 0.2 | g/L |
| 10. H₂SO₄ | 5 | mL |
| C. glycerol | 8% | (V/V) |

D. Feed medium 50% glycerol (1 L) + 12 mL/L PTM1
E. Induced medium 100% methanol (1 L) + 12 mL/L PTM1

| F. Dissolved O₂ (DO) | >20% | |
|---|---|---|
| G. pH | 5.0-5.8 | |
| H. Temperature | 20-30° | C. |

(3) Second High-Density Fermentation Medium and Culture Conditions for Pichia pastoris in a Fermenter:

### Preparation of a Solution A:

YPD medium (50 mL):

| | | |
|---|---|---|
| 1. Yeast extract | 0.5 | g |
| 2. peptone | 1 | g |
| 3. Glucose | 1 | g |

The YPD medium was used for culturing a primary seed culture of Pichia pastoris.

### Preparation of a Solution B:

BSM medium (1 L):

| | | |
|---|---|---|
| 1. 98% glycerol | 63 | g/L |
| 2. ddH₂O (Ultrapure water) | 912 | mL |
| 3. CaSO₂•2H₂ O | 0.46 | g/L |
| 4. MgSO₄•7H₂ O | 5.84 | g/L |
| 5. K₂SO₄ | 7.34 | g/L |
| 6. (NH₄)₂SO₄ | 9 | g/L |

The BSM medium was used for culturing a secondary seed culture of Pichia pastoris and served as a primary nutrient medium for yeast culture in a bioreactor.

### Preparation of a Solution C:

### Preparation of a Sodium Hexametaphosphate (HMP) Solution (150 mL):

| | | |
|---|---|---|
| 1) (NaPO₃)₆(Sodium hexametaphosphate) | 30 | g |
| 2) ddH₂O (ultrapure water) | 150 | mL |

### Preparation of a Solution D:

PTM1 solution (1 L):

| | | |
|---|---|---|
| 1. CuSO₄•5H₂ O | 6 | g/L |
| 2. KI | 0.08 | g/L |
| 3. MnSO₄•H₂O | 3 | g/L |
| 4. Na₂MoO₄•2H₂ O | 0.2 | g/L |
| 5. H₃BO₃ | 0.02 | g/L |
| 6. CoCl₂ | 0.5 | g/L |
| 7. ZnCl₂ | 20 | g/L |
| 8. FeSO₄•7H₂O | 65 | g/L |
| 9. Biotin | 0.2 | g/L |
| 10. H₂SO₄ | 5 | mL |

### (4) Plasmid Extraction

### 1) Small-Scale Plasmid Extraction

Single colonies were picked and inoculated into 2 mL of an LB medium including a corresponding antibiotic, and cultured overnight at 37° C. under shaking. The next day, 1.5 mL of a resulting culture was collected in an Eppendorf tube and centrifuged to produce a cell pellet and a supernatant, and the supernatant was discarded. The cell pellet was placed in an ice bath. 100 μL of a solution I (50 mM glucose, 25 mM Tris-HCl, and 10 mM EDTA, pH 8.0) was added, shaking was conducted for thorough mixing, and incubation was conducted at room temperature for 5 min. 200 μL of a solution II (0.2 N NaOH and 1% SDS) was added, inverting was repeated for thorough mixing, and incubation was conducted in an ice bath for 5 min. 150 μL of a solution III (3 M NaAc, pH 4.8) was added, gentle shaking was conducted for thorough mixing, and incubation was conducted in an ice bath for 5 min. 450 μL of redistilled phenol/chloroform (1:1) was added, and thorough mixing was conducted. Centrifugation was conducted at 12,000 rpm for 10 min. A resulting upper aqueous phase was carefully transferred to pre-cooled absolute ethanol in a volume 2 times the volume of the upper aqueous phase, and a resulting mixture was placed in a −20° C. refrigerator for 2 h and centrifuged at 12,000 rpm for 15 min. A resulting ethanol phase was removed, and 500 L of 70% ethanol was carefully added for washing to remove salts. Centrifugation and vacuum-drying were conducted. 18 L of TE and 2 L of an RNase solution were added, and incubation was conducted at 37° C. for 1 h. A resulting sample was for enzyme digestion analysis and further cloning.

### 2) Large-Scale Plasmid Extraction

500 mL of a strain solution produced after culturing overnight was collected and centrifuged at 5,000 rpm and 4° C. for 10 min. Washing was conducted with 100 mL of STE. Then, centrifugation was conducted, and a resulting cell pellet was collected. 18 mL of a solution I (50 mM glucose, 25 mM Tris-HCl, and 10 mM EDTA, pH 8.0) and 2 mL of a lysozyme (10 mg/mL, 1% SDS) were added successively, and incubation was conducted at room temperature for 10 min. 40 mL of a solution II (0.2 N NaOH and 1% SDS) was added, thorough mixing was conducted gently (until a resulting solution was transparent), and incubation was conducted in an ice bath for 5 min. Then 20 mL of a solution III (3 M NaAc, pH 4.8) was added, thorough mixing was conducted, and incubation was conducted in an ice bath for 10 min. Extraction was conducted twice with a phenol-chloroform (volume ratio: 1:1) solution. Pre-cooled absolute ethanol was added in a two-fold volume, and incubation was conducted overnight at −20° C. Centrifugation was conducted at 12,000 rpm for 15 min, and a resulting supernatant was discarded. A resulting precipitate was washed 1 time to 2 times with 70% ethanol, then vacuum-dried, and dissolved in 3 mL of TE. 10 μL of an RNase solution (10 mg/mL) was added, and incubation was conducted at 37° C. for 30 min. A resulting sample was loaded onto a Sepharose 2B chromatographic column (1×10 cm, pre-equilibrated with TE). A first peak (high-molecular-weight DNA) was collected with TE (pH 7.6) as an eluent. The volume of a collected product was measured. Pre-cooled absolute ethanol in a volume 2 times the volume of the collected product and 3 M NaAc (pH 5.2) in a volume 1/10 of the volume of the collected product were added. A resulting mixture was thoroughly mixed, placed overnight in a −20° C. refrigerator, and centrifuged at 12,000 rpm for 15 min. A resulting supernatant was discarded. A resulting precipitate was washed 1 time to 2 times with 70% ethanol, vacuum-dried, and dissolved in 1 mL of TE. An appropriate amount of the resulting sample solution was taken and tested for a DNA concentration using an ultraviolet spectrophotometer at 260 nm. Recombinant plasmid DNA obtained from the large-scale extraction could be used for the protoplast-mediated transformation into a Pichia pastoris host strain and the long-term cryopreservation in the present disclosure.

(5) Preparation of E. coli Competent Cells and Transformation of the Recombinant Plasmid

An E. coli host strain was inoculated into 2 mL of an LB medium and cultured overnight at 37° C. under shaking. The next day, 500 L of a resulting culture was inoculated into 50 mL of an LB medium and cultured for 1 h at 37° C. and 300 rpm under shaking until a cell density reached OD₆₀₀=0.5. Centrifugation was conducted for 5 min at 5,000 rpm and 4° C. A resulting supernatant was decanted. A resulting cell pellet was resuspended in 25 mL of a cold solution including 100 mM of CaCl₂ and 10 mM of Tris-HCl (pH 7.4), placed in an ice bath for 30 min to 60 min, and then centrifuged for 5 min at 5,000 rpm and 4° C. A resulting supernatant was decanted. A resulting cell pellet was resuspended in 2 mL of a cold calcium chloride solution, placed in an ice bath for 40 min, and then could be used for transformation.

200 μL of the freshly-prepared E. coli competent cell suspension was added to an Eppendorf tube, and 10 μL of a recombinant plasmid DNA-containing ligation solution were added. Incubation was conducted in an ice bath for 30 min, and a heat shock was conducted at 42° C. for 2 min. 500 μL of an LB medium was added, and incubation was conducted for 1 h on a shaker at 37° C. under gentle shaking. Centrifugation was conducted at 10,000 rpm for 10 s. Most of the resulting LB medium supernatant was discarded with approximately 200 μL left. A resulting bacterial suspension was thoroughly mixed gently with a pipette tip, and then divided into two portions of 50 μL and 150 μL. Each portion was coated on an LB agarose plate including 50 g/mL of ampicillin, and cultured in a 37° C. incubator for 8 h to 16 h.

### Example 2

Design of a Mature HSA Gene for High-Level Expression in Komagataella phaffii Host Strains

This embodiment pertains to the optimized design of the HSA mature gene. As shown in SEQ ID NO: 1, the characteristic gene sequence has a nucleic acid length of 1755 bp, which encodes a 585-amino-acid sequence corresponding to the characteristic protein sequence (see SEQ ID NO: 2) that represents one of the longest functionally active genes ever chemically synthesized internationally.

The core design principle of this invention is to utilize the Komagataella phaffii integrated expression system, currently recognized as the most advanced microbial expression platform worldwide, as the host for expressing the target gene. The gene is driven by the potent promoter of the alcohol oxidase (AOX1) gene from Komagataella phaffii, while the codons of the human serum albumin (HSA) structural gene are replaced with preferred codons that align with the high-expression specificity of AOX1.

This invention involves a novel and specific optimization design for the HSA gene, utilizing three core molecular biology databases: 1. The International Nucleic Acid Sequence Database (GenBank/EMBL/DDBJ); 2. The Swiss Protein Sequence and Annotation Database (Swiss-PROT); 3. The Protein Data Bank (PDB) provided by the U.S. National Laboratory of Brookhaven. Multiple computer software packages (including GENESIS and PROSIS developed by the Genetic Computer Group at the University of Wisconsin, Caltec software by the California Institute of Technology, DNASIS and PROSIS by Pharmacia of Sweden, and other programs) were employed for comprehensive auxiliary analysis. The novel design of the HSA gene was performed on a SGI R4400 workstation, adhering to the following principles:

1. In step S100, when encoding the optimized gene for human serum albumin, the preferred codons of Pichia pastoris alcohol oxidase (AOX1) gene were selected, with these preferred codons constituting 90% of the total codons in the optimized gene.

2. In step S100, the optimized gene encoding human serum albumin was sequentially inserted with three restriction enzyme sites (SalI, HindIII, XbaI) from 5′ to 3′ direction, which divided the optimized gene into four large fragments in a relatively balanced way.

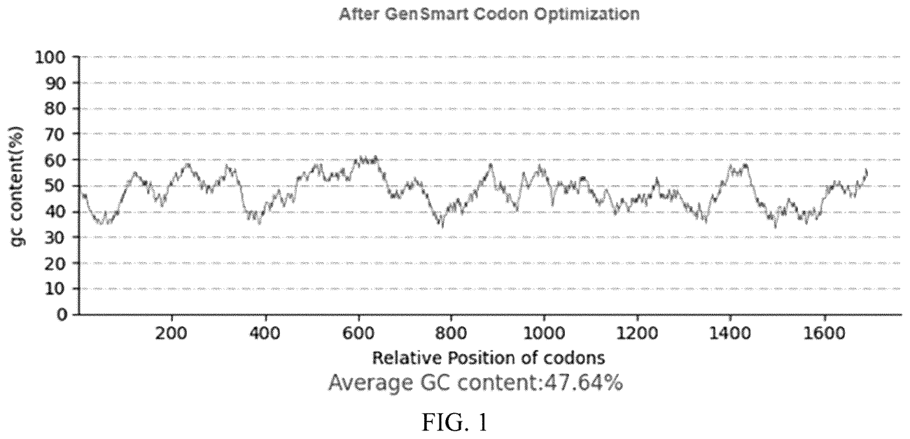

3. In step S100, the optimized gene encoding human serum albumin was engineered to reduce consecutive G-C pairs and enhance A-T pairs preferred by Komagataella phaffii, achieving a balanced base distribution within the gene. This adjustment brought the GC content within the optimized gene to 45-50% (as shown in FIG. 1).

4. In step S100, the optimized gene encoding human serum albumin further includes an expression reading cassette constructed as follows:

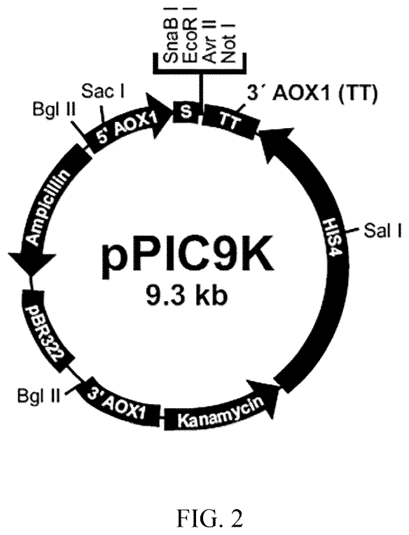

A 5′ restriction endonuclease site BamHI is inserted into a 5′ regulatory region (promoter region) of the AOX1 gene. A 10-deoxynucleotide oligonucleotide CCAAACGATG as shown in SEQ ID NO: 9 (including a Kozak sequence for eukaryotic genes, which is AXXATG) is ligated. A yeast α-mating factor leader peptide sequence (including 85 amino acids) derived from Saccharomyces cerevisiae is then ligated. The mature HSA gene was inserted between the EcoRI and NotI sites of the pPIC9K recombinant expression vector's polyclonal site (see FIG. 2).

The coding sequence for lysine and arginine (-Lys-Arg-) was inserted at the 5′ end of the target gene, following the EcoRI restriction site, as shown in SEQ ID NO: 4. Additionally, a double stop codon (TAATAG) was inserted before the Not I restriction site at the 3′ end, as shown in SEQ ID NO: 5. These double stop codons enhance the translation termination signal to prevent read-through transcription during gene expression.

### Example 3

### Construction of Recombinant Expression Vector HSA-pPIC9K

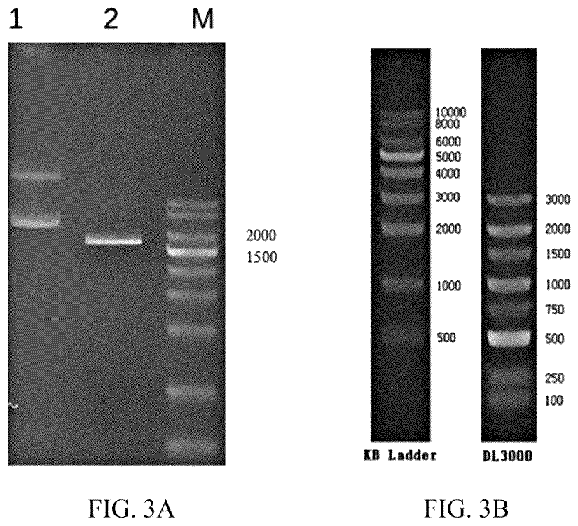

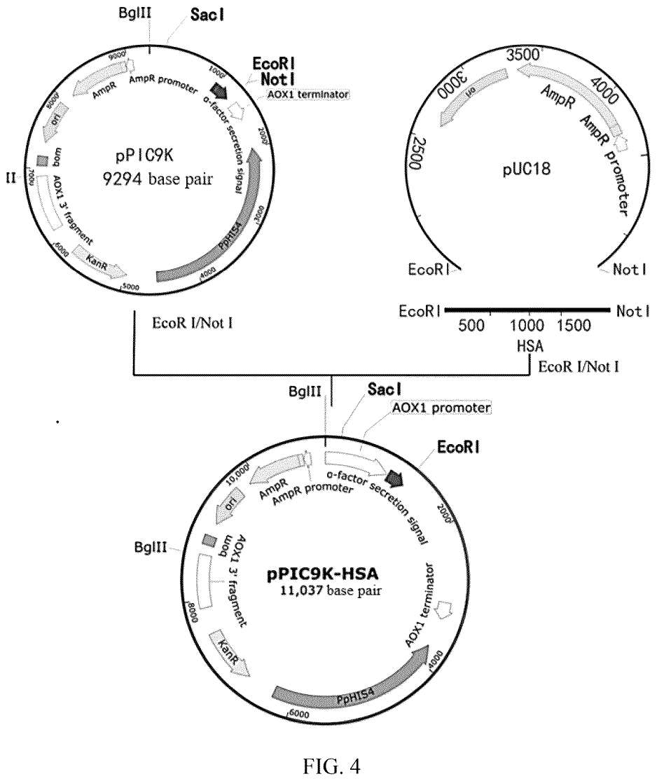

The mature gene encoding human serum albumin (1755 bp) synthesized through chemical total synthesis in Example 1 was digested with EcoRI-SmaI enzymes to remove it from the pUC18 cloning vector. The fragment was identified via 1% agarose gel electrophoresis, with results shown in FIG. 3 (Enzyme digestion results: A: Lane 1 represents the recombinant plasmid; Lane 2 represents the recombinant plasmid digested with EcoRI and SmaI; M lane represents the molecular weight standard KB Ladder. B: Two lanes respectively represent the molecular weight standard KB Ladder and DL3000), consistent with expected results. The KpnI restriction site in the pUC18 cloning vector was modified to NotI. The mature gene encoding human serum albumin (1755 bp) synthesized through chemical total synthesis was digested with EcoRI-NotI enzymes to remove it from the pUC18 cloning vector, and the corresponding size gel bands were used with a DNA recovery kit to isolate the target gene fragment. The HSA fragment digested with EcoRI-NotI from the pUC18 cloning vector was cloned into the Komagataella phaffii (formerly named Pichia pastoris) expression vector pPIC9K, which was also digested with the corresponding enzymes. The recombinant expression vector HSA-pPIC9K (containing the mature HSA gene, see SEQ ID NO: 3) was constructed, with the specific plasmid construction flowchart shown in FIG. 4. The ligation reaction system was:

| | |
|---|---|
| HSA DNA fragment (60 ng) | 4 μl |
| T4 DNA ligase (2 U) | 1 μl |
| pPIC9K vector (240 ng) | 5 μl |
| ddH₂0 | 6 μl |
| 5 × ligase buffer | 4 μl |

The reaction was carried out at 16° C. in 20 l of reaction system overnight.

The ligating solutions containing the recombinant plasmid HSA-pPIC9K were added to two concentrations of transformed competent E. coli JM109 host cells (4 μl and 8 μl) respectively. The cultures were spread on LB plates (containing 20 μg/ml ampicillin) and incubated overnight in a 37° C. incubator. Subsequently, 18 randomly selected transformant colonies containing the HSA-pPIC9K plasmid were transferred to 2 ml LB medium (containing 20 μg/ml ampicillin). After 6-8 hours of shaking, the plasmid DNA was rapidly extracted using alkaline denaturation. The DNA was then digested with EcoRI-NotI enzymes and analyzed by 1% agarose gel electrophoresis to identify HSA-containing recombinants of corresponding sizes. Five clones containing HSA-specific EcoRI-NotI fragments were successfully isolated. The identified E. coli host cells containing HSA-pPIC9K plasmids were then transformed to obtain large quantities of the recombinant plasmid using the large-scale plasmid extraction method. The extracted plasmid was stored for long-term preservation or later transformed into Komagataella phaffii host cells. Detailed design specifications and sequencing results of the HSA-pPIC9K recombinant plasmid are shown in FIG. 4, FIG. 5, FIG. 6, and FIG. 7. The sequence alignment analysis of sequencing result (SEQ contig) are exhibited in SEQ ID NO: 6, SEQ ID NO: 7 and SEQ ID NO: 8.

### Example 4

Transformation of Recombinant Plasmid HSA-pPIC9K into Komagataella phaffii Host Cells by Biorad Gene Pulser Xcell System

The HSA-pPIC9K recombinant plasmid constructed in Example 2 was linearized using restriction enzymes SacI or Bgl II. The linearized plasmid was then transformed into Komagataella phaffii host strains (GS115 or CBS7435) via electroporation. The detailed procedure is described as follows:

### (I) Preparation of the Culture Medium

1. BMGY medium: 10 g yeast powder and 20 g peptone dissolved in 700 mL deionized water, then subjected to moist heat sterilization for 20 minutes. After cooling, 100 mL of 1M potassium phosphate buffer (pH 6.0), 100 mL of 10×YNB, 2 mL of 500×B, and 100 mL of 10×GY were added, and the mixture was stored at 4° C.

2. BMMY medium: Yeast powder 10 g, peptone 20 g, dissolved in 700 mL deionized water, moist heat sterilized for 30 min, cooled, then added 100 mL 1M pH6.0 potassium phosphate buffer, 100 mL 10×YNB, 2 mL 500×B, 100 mL 10×M, stored at 4° C.

3. MD medium: 100 mL 10×YNB, 2 mL 500×B, 100 mL 10×D added to 800 mL sterilized water, stored at 4° C.

4. 1M potassium phosphate buffer (pH 6.0): 132 mL of 1M K₂HPO₄ and 868 mL of 1M KH₂PO₄, pH 6.0, sterilized by moist heat, stored at 4° C.

5. 10×YNB: Dissolve 134 g YNB (ammonium sulfate included) in 1000 mL deionized water, filter for sterilization, and store at 4° C.

6. 500×B: 20 mg biotin dissolved in 100 mL deionized water, filtered and sterilized, then stored at 4° C.

7. 10×M: 5 mL methanol mixed with 95 mL deionized water, filtered and sterilized, stored at 4° C.

8. 10×GY: 100 mL glycerol mixed with 900 mL deionized water, sterilized by moist heat, and stored at 4° C.

9. 10×D: 100 g glucose dissolved in 1000 mL deionized water, filtered for sterilization, and stored at 4° C.

10. 1M sorbitol: 18.2 g D-sorbitol is dissolved in 100 mL deionized water, filtered and sterilized, and stored at 4° C.

### (II) Preparation of the Competent Cells

1. Select a single colony of Komagataella phaffii (GS115 or CBS7435) and inoculate it into a 50 ml Erlenmeyer flask containing 5 ml YPD medium. Incubate at 30° C., 250-300 r/min overnight.

2. 100-500 μl of the culture was inoculated into a 2 L triangular flask containing 500 ml fresh medium, and incubated at 28-30° C., 250-300 r/min overnight until OD600 reached 1.3-1.5.

3. The cell culture was centrifuged at 4° C. for 5 minutes at 1500 g, and the cell pellet was resuspended in 500 ml ice-precooled sterile water.

4. Centrifuge as step 3, and resuspend the bacterial precipitate with 250 ml of ice-precooled sterile water;

5. Centrifuge the cells according to step 3, and resuspend the cells in 20 ml of 1M sorbitol solution pre-cooled with ice.

6. Centrifuge the cells according to step 3, and resuspend the cells in 1 ml of 1M sorbitol solution pre-cooled with ice, the final volume is about 1.5 ml.

7. Note: The product may be frozen in 80 μl portions, but this will affect its conversion efficiency (within 2 weeks).

### (III) Electroporation Transformation

1. Add 80 μL of the prepared Komagataella phaffii competent cells and 5-20 μg of linearized DNA (dissolved in double distilled water, 5-10 μL) to a 1.5 mL pre-cooled centrifuge tube and mix thoroughly. Transfer the mixture to a 0.2 cm transformation cup that has been pre-chilled in an ice bath.

2. The ice bath was filled with the conversion cup containing the conversion mixture for 5 minutes.

3. Based on the data from the electrical converter, referencing other literature, and through repeated experimentation, the appropriate parameters such as voltage, current, and capacitance were determined. The Biorad Gene Pulser Xcell System was performed using these optimized parameters. The preferred parameters for this invention are: voltage 1500-1800V; capacitance 25 μF; resistance 200-400Ω; and shock duration 4-10 msec.

4. Immediately after the pulse, add 1 mL of 1M sorbitol solution in ice bath into the conversion cup, then transfer the conversion solution into a new 1.5 mL centrifuge tube.

5. The cells were cultured in a 28° C. shaking incubator for 1-2 hours.

6. Take 50-200 μL of transformation liquid of the host strain of Komagataella phaffi GS115 or CBS7435 and spread it on MD plates.

7. The cells were cultured in 28° C. incubator for 3-4 days until many single clone positive colonies (yeast transformants) were formed.

### (IV) Screening of Multiple Copy Transformants

1. Wash the MD plates with 2 mL sterile water to remove yeast transformants, then transfer the strain suspension to a centrifuge tube using a pipette.

2. The obtained strain suspension was spread on the YPD plates containing G418 resistance, 100 μL of strain suspension was spread on each plate; the content of G418 antibiotic was prepared in different concentration gradient: 0.25%, 0.5%, 1.0%, 1.25%, 1.5%, 1.75%, 2.0% etc.

3. The plates were incubated in 28° C. incubator for 4-12 days, and different G418 resistant white single colonies were obtained (i.e. different copy number of Komagataella phaffii transformants).

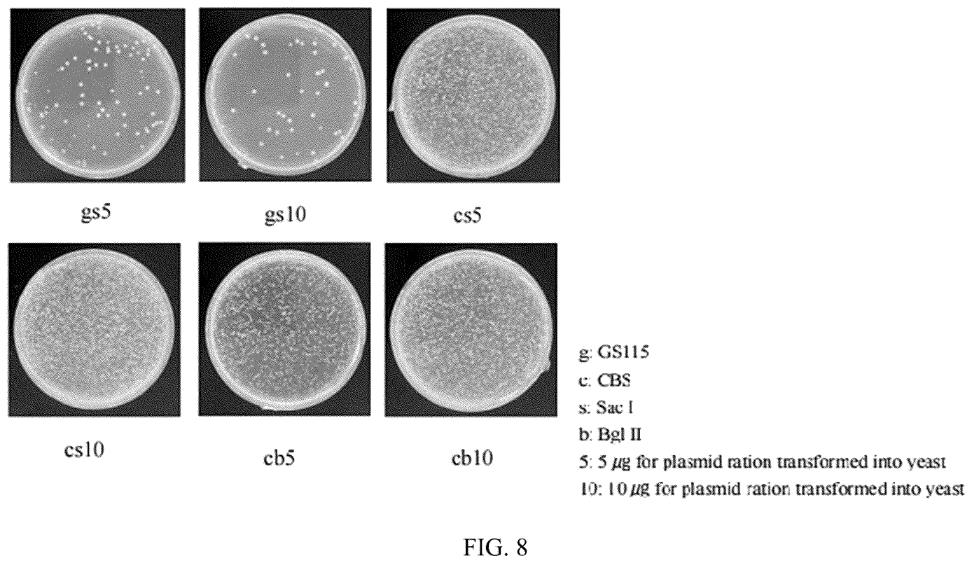

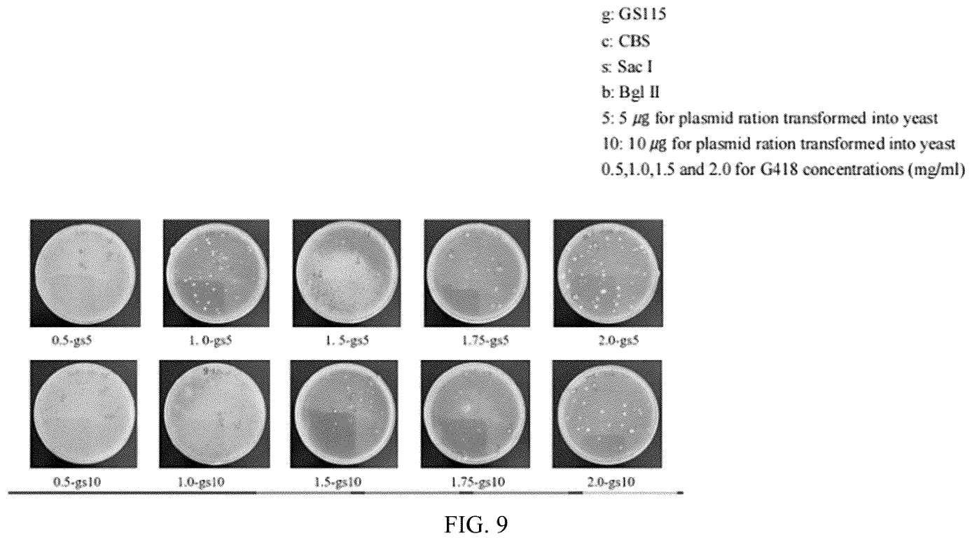

The experimental results of electrochemical conversion of host strain GS115 or CBS7435 in this embodiment are partially presented in FIG. 8, FIG. 9, FIG. 10, and Tables 1 and 2.

TABLE 1
Experimental results of multi-copy Pichia pastoris
GS115 engineered strains

| | Competent | G418 | Linearization | Plasmid for |
|---|---|---|---|---|
| | cell | resis- | enzyme | electroporation |
| No. | identifier | tance | SacI/BglII | (μg) |
| G1 | g | 0.50 | S | 5 |
| G2 | g | 0.50 | S | 5 |
| G3 | g | 0.50 | S | 5 |
| G4 | g | 0.50 | S | 10 |
| G5 | g | 0.50 | S | 10 |
| G6 | g | 0.50 | S | 10 |
| G7 | g | 1.00 | S | 5 |
| G8 | g | 1.00 | S | 5 |
| G9 | g | 1.00 | S | 5 |
| G10 | g | 1.00 | S | 10 |
| G11 | g | 1.00 | S | 10 |
| G12 | g | 1.00 | S | 10 |
| G13 | g | 1.75 | S | 5 |
| G14 | g | 1.75 | S | 5 |
| G15 | g | 1.75 | S | 5 |
| G16 | g | 1.75 | S | 10 |
| G17 | g | 1.75 | S | 10 |
| G18 | g | 1.75 | S | 10 |
| G19 | g | 1.50 | S | 5 |
| G20 | g | 1.50 | S | 5 |
| G21 | g | 1.50 | S | 5 |
| G22 | g | 1.50 | S | 10 |
| G23 | g | 1.50 | S | 10 |
| G24 | g | 1.50 | S | 10 |

Note:
In Table 1, G418 resistance of 0.25 indicates that a host strain carries one copy of the exogenous gene, and so on.

TABLE 2
Experimental results of screening of multi-copy
Pichia pastoris CBS7435 engineered strains

| | Small-scale | Competent | G418 | Linearization | Plasmid for |
|---|---|---|---|---|---|
| | induction | cell | resis- | enzyme | electroporation |
| No. | batch | identifier | tance | SacI/BglII | (μg) |
| C1 | 1 | c | 1.0 | S | 5 |
| C2 | 2 | c | 0.25 | S | 5 |
| C3 | 2 | c | 0.25 | S | 5 |
| C4 | 2 | c | 0.5 | S | 5 |
| C5 | 2 | c | 0.5 | S | 5 |
| C6 | 2 | c | 0.5 | S | 5 |
| C7 | 2 | c | 1.0 | S | 5 |
| C8 | 2 | c | 1.0 | S | 5 |
| C9 | 2 | c | 1.0 | S | 5 |
| C10 | 3 | c | 1.0 | S | 5 |
| C11 | 3 | c | 1.0 | S | 5 |
| C12 | 4 | c | 1.25 | S | 5 |
| C13 | 4 | c | 1.25 | S | 5 |
| C14 | 4 | c | 1.25 | S | 5 |
| C15 | 4 | c | 1.5 | S | 5 |
| C16 | 4 | c | 1.75 | S | 5 |
| C17 | 4 | c | 1.75 | S | 5 |
| C18 | 4 | c | 1.75 | S | 5 |
| C19 | 5 | c | 0.5 | S | 5 |
| C20 | 5 | c | 0.5 | S | 10 |
| C21 | 5 | c | 0.5 | S | 10 |
| C22 | 5 | c | 0.5 | S | 10 |

Note:
In Table 2, G418 resistance of 0.25 indicates that a host strain carries one copy of the exogenous gene, and so on.

### Example 5

### Small-Scale Induction Experiment of Recombinant Human Serum Albumin High Expression Engineering Strain

From the transformants containing recombinant plasmid HSA-pPIC9K with varying copy numbers screened by the electroporation method in Example 3. The 12 clones were randomly selected from YPD plates. Single colonies were inoculated into 4 ml BMGY liquid medium test tubes and incubated overnight on a shaker at 28° C. with 200-250 rpm. The strain suspensions were centrifuged at 1500-3000 g for 5 minutes at room temperature, followed by collection of strain cells. The cells were resuspended in 4 ml of original culture volume of BMMY medium and incubated on a shaker at 28° C. with 250-300 rpm for 36-48 hours. After centrifugation at 5000 rpm for 10 minutes, 30 μL of supernatant was collected, vacuum-dried, and subjected to 1000 SDS-PAGE electrophoresis for identification. High-expression clones were identified using pPIC9K empty vector expression as control. The selected high-expression clones were then cryopreserved at 15% glycerol for use as engineered strains in subsequent expression experiments.

For pilot-scale induction experiments using the engineered high-expression strains, the Komagataella phaffii GS115 strains follow this protocol: First, the glycerol-Streptococcus strain is inoculated into YPD liquid medium for reactivation. Then, 1 ml of seed culture is transferred to 50 ml BMGY medium and incubated at 28° C. with 200-250 rpm for 18-24 hours. Next, 4 ml of 4% seed culture is added to 1000 ml BMGY medium, which is then incubated at 28° C. with 250-300 rpm for 24-30 hours. After centrifugation at 5000 rpm for 5 minutes, the supernatant is discarded. The bacterial cells are resuspended in 500 ml BMGY induction medium and cultured at 28° C. with 250-300 rpm for 3-4 days, with 100% methanol added every 24 hours to maintain 0.5% protein concentration. At this stage, the engineered Pichia pastoris cells typically achieve a cell density of 18-20 OD₆₀₀, with most exogenous protein expressed in the liquid medium. The fermentation medium is then centrifuged at 10000 rpm for 20 minutes at 4° C., and the supernatant containing abundant HSA expression products is collected. SDS-PAGE electrophoresis is performed on the supernatant samples to analyze and identify the expression levels of recombinant HSA.

For the pilot-scale induction experiments of the engineered high-expression strains, if using Komagataella phaffii CBS7435, the experimental protocol will follow the sequence: glycerol broth→YPD→YPD→BSM→BSM, with sample volumes comparable to those of the GS115 strains.

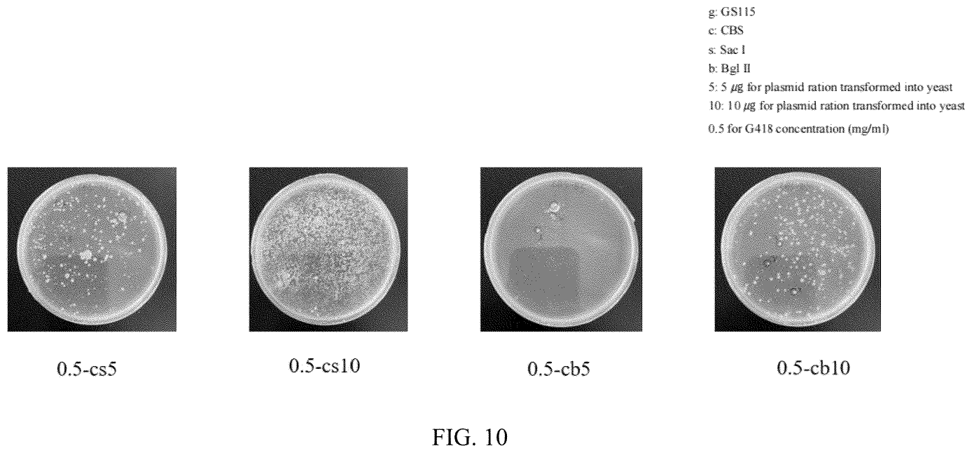

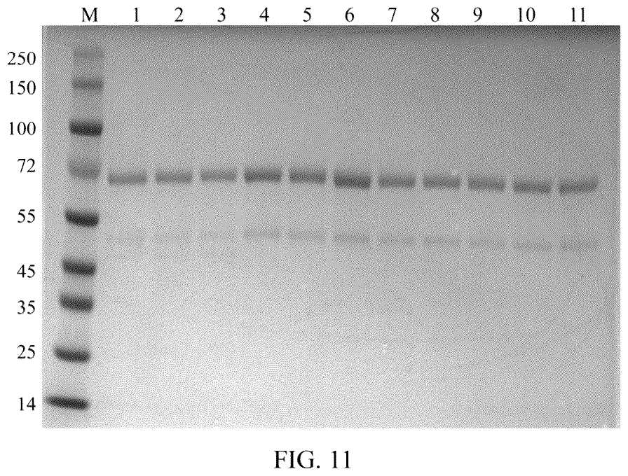

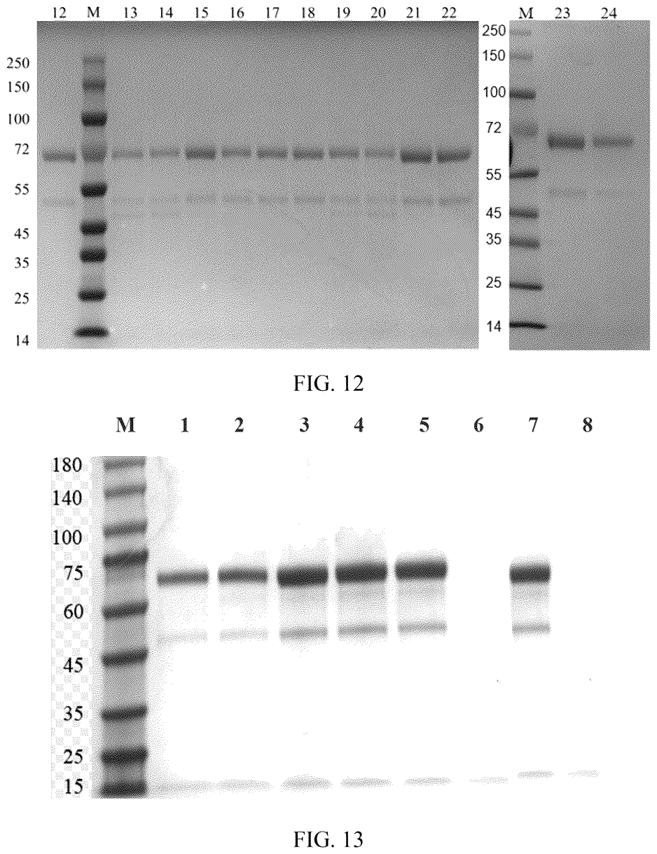

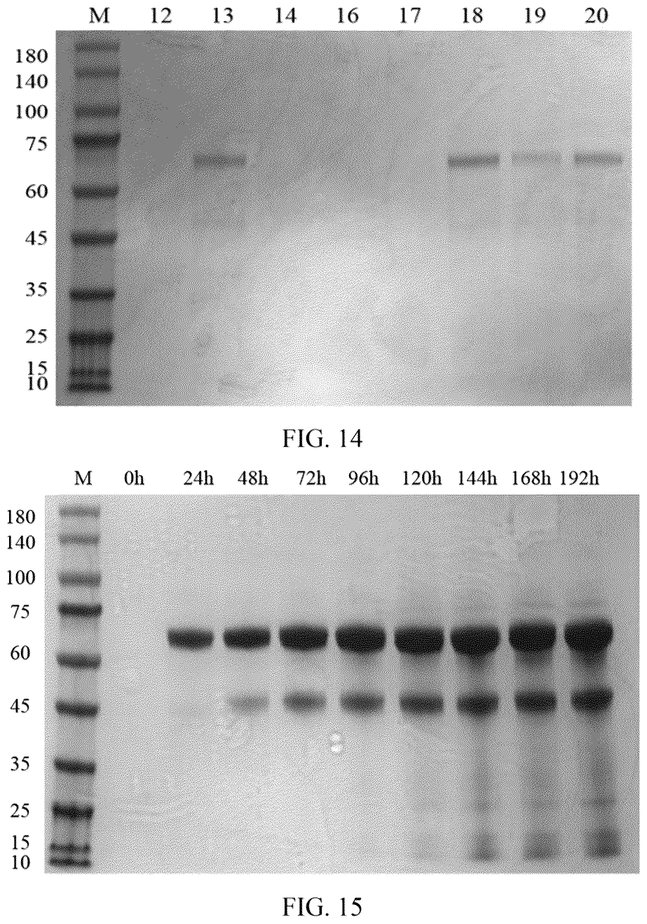

The small-scale induction experiments of selected Komagataella phaffii GS115 or CBS7435 strains are partially presented in FIG. 11, FIG. 12, FIG. 13, and FIG. 14.

### Example 6

High-Density Fermentation of Komagataella phaffii Engineered Strains in 50 L, 500 L and 2000 L Fermenters

In Example 4, the engineered strains exhibiting distinct expression bands in the pilot-scale induction experiment were further subjected to high-density cell culture fermentation using fed-batch fermentation (Fed-batch fermentation) in 5 L, 50 L, 500 L, and 2000 L fermenters. This was conducted to optimize the Komagataella phaffii BSM fermentation process and validate the pilot-scale fermentation performance of the electroporated GS115 or CBS7435 engineered strains. The detailed implementation protocol is as follows:

### (I) Preparation Phase of the Experiment

### 1. Culture Medium Preparation

The preparation of the fermentation substrate medium for this invention is shown in Table 3 below.

TABLE 3
Preparation of the fermentation substrate medium (6 L)

| Reagent | Formula (g/L) | | Preparation volume: 6 L | |
|---|---|---|---|---|
| H₃PO₄ | 26.7 | ml | 160 | ml |
| CaSO₄•2H₂O | 0.47 | | 2.8 | |
| K₂SO₄ | 9.1 | | 55 | |
| MgSO₄•7H₂O | 7.5 | | 45 | |
| KOH | 4.13 | | 25 | |
| 98% glycerol | 40 | | 240 | |
| Defoaming agent | 0.5 | ml | 3 | ml |

When preparing the fermentation substrate medium, store the measuring cups or beakers for phosphoric acid and glycerol separately. Other inorganic salts should be dissolved sequentially in about 2 L of purified water, adding each reagent after the previous one is fully dissolved. The final KOH must also be dissolved separately and not mixed with the inorganic salts.

### 2. Electrode Calibration

2.1 pH Electrode Calibration: Prepare standard pH electrode solutions (pH6.86 and 4.00), washing bottles, and paper towels. Connect the pH electrode to the fermentor's electrode wire. Rinse the electrode with the washing bottle, absorb excess water with a paper towel, then insert it into the pH6.86 solution. Once the pH reading stabilizes, press the calibration button to set the zero point. Remove the electrode, rinse again with the washing bottle, absorb water with a paper towel, and insert it into the pH4.00 solution. After the pH reading stabilizes, press the calibration button to set the slope. Repeat this process three times. The pH readings should match the solution values, completing the electrode calibration.

2.2 Dissolved Oxygen Electrode Calibration: After connecting the dissolved oxygen electrode to the fermentor's electrode wire and cleaning it, immerse the electrode in saturated anhydrous sodium sulfite solution. Let it stand for 15 minutes until the dissolved oxygen reading stabilizes. Then press the calibration button to set the zero point. Before inoculating the fermentation, calibrate the electrode at 100% slope by maintaining 100 rpm agitation, 0.5 vvm aeration, and 0.05 MPa tank pressure. This completes the dissolved oxygen electrode calibration.

2.3 Feeding procedure: sequentially introduce phosphoric acid and glycerol into the fermentor, followed by inorganic salt solution, and finally KOH. Add purified water to make up to 5 L (considering the fermentor's weight of approximately 5 Kg, the volume should be around 6 L after sterilization). After feeding, the pH value should be maintained below 2.

2.4 Prepare 1 L of PTM1 solution. Store it in a sealed container and keep away from light.

2.5 100% analytical grade methanol 3 L.

2.6 50% glycerin feedstock: 800 ml

We weighed 400 g 98% glycerol, added purified water to make up to 800 ml, dissolved and poured into a 1 L feeding bottle, sterilized at 121° C. for 30 minutes, and cooled for later use.

(II) Fermentation Process Development of the Engineered Strain of Komagataella phaffii

### 1. Seed Preparation:

For the primary seed preparation, take the glycerin-cultured strains designated as CBS7435-C1-C50 or GS115-G1-G30, transfer 100 μl to 20 ml of the strains suspension into 100 ml of YPD medium, and incubate at 28° C. with a 200 rpm shaker for 24 hours. Transfer 5 ml of the strains suspension to 480 ml/2 L Erlenmeyer flasks containing YPD medium as the secondary seed preparation, and incubate at 28° C. with a 200 rpm shaker for 24 hours. Transfer 5 ml of the primary seed preparation to 480 ml/2 L Erlenmeyer flasks containing BSM medium (two flasks in total) as the secondary seed preparation, and incubate at 28° C. with a 200 rpm shaker for 24 hours. The OD₆₀₀ of the secondary seed preparation should be between 20 and 40 before inoculation.

Transfer the inoculated strains into the fermenter.

Before inoculation, ensure the fermenter temperature is 30° C. and set the pH to 5.0 using ammonia water for automatic pH adjustment. Transfer the seed solution into the fermenter via flame loop inoculation to complete the process. For flame loop inoculation, wrap the fermenter's inoculation port with an alcohol swab and ignite it. Reduce the air inlet valve while opening the exhaust valve to maintain tank pressure at approximately 0.02 MPa. Gently open the inoculation port with specialized tools, vent air until no audible sound is heard, then fully rotate the port. Remove the sealing tape from both the seed culture bottle and PTM1 container, discard the sealing film above the flame loop, and pour the seed culture and PTM1 into the tube. Never mix the seed culture with PTM1 prior to inoculation.

### 3. Fermentation Stage of Recombinant Human Serum Albumin:

### (III) the Basic Culture Phase of the Engineered Strains

The fermentation of the engineered strains begins, and the SCADA process control software is activated to record data, including fermentation time and batch numbers, as well as to start spectrum recording. From the moment the seed bacteria are introduced into the fermentor, relevant fermentation data (such as time, temperature, agitation, pH, dissolved oxygen (DO), air flow rate, and tank pressure) are recorded every 4 hours. The control parameters are set as follows: use ammonia water to maintain pH at 5.5±0.1, temperature at 28±1° C., DO above 30, air flow rate at 2 vvm, and tank pressure at 0.03-0.04 Mpa. After a DO rebound (where the DO value increases by more than 10 without any operation on the fermentor, agitation, or tank pressure), the system enters the transition culture phase. During the rebound, the OD₆₀₀ sample should be around 50.

### (IV) Transition Culture Phase of the Engineered Strains

The transition culture phase of the engineered strains is aimed at further increasing the biomass of the engineered strains. Fermentation data is recorded hourly. During the transition culture phase, 50% glycerol is fed. After the DO rebound, immediately add 50% glycerol (12 ml/L of PTM1 solution containing biotin, approximately 10 ml). At the same time, adjust the agitation to the maximum speed specified by the fermentor, set the air flow rate to 2 vvm, and adjust the tank pressure to 0.04-0.08 Mpa. Under these conditions, the feeding rate should be controlled to maintain the DO value at 30±10. Perform at least one DO-Spike every hour to ensure the response time of the DO-Spike is less than 60 seconds. The feeding cycle is approximately 4-5 hours. During glycerol feeding, adjust the pH setting every hour on average to make it the desired induction pH value after glycerol feeding (for this fermentation, the pH during the induction phase is the same as during the culture phase, both at 5.7, so no pH adjustment is needed). When the transition culture phase ends, the OD₆₀₀ is around 100. At this point, take a sample of the wet weight of the bacterial solution and retain it for 0 hours. Thereafter, each sample taken should be tested for wet weight without testing OD₆₀₀. Stop the glycerol feeding and enter the methanol induction phase.

### (V) Induction Expression Phase of Engineered Strains

After ceasing glycerol supplementation, observe the dissolved oxygen (DO) rebound and prepare to add methanol (containing 12 ml/L PTM1). Add 4 g/L methanol in one batch (total 24 g methanol in this experiment, which can be delivered through a disposable syringe via feeding gasket). Once the added methanol is depleted (DO rebound occurs), initiate methanol fed-batch using DO-Spike method with optimal response time under 60 seconds, maintaining DO levels between 10-30. Simultaneously, adjust the temperature to the specified project temperature (24° C. in this experiment) and control pH to the designated pH5.7. During the engineered yeast induction phase, perform DO-Spike every 12 hours, recording response times and fermentation data. Sample pH and wet weight of engineered yeast every 12 hours, centrifuge, and transfer 100 μl of supernatant to electrophoresis buffer. Boil at 100° C. for 5 minutes before storing at −20° C. Other supernatants are stored in a 2-8° C. refrigerator for testing or electrophoresis analysis. Ensure proper sample labeling during sampling. Throughout the Komagataella phaffii engineered yeast induction phase, maintain methanol concentration in the culture medium within 0.2-0.9% range.

(VI) Terminate the Fermentation of this Batch

The project specified the fermentation time for the engineered Komagataella phaffii strain in the fermenter (the induction period was approximately 200 hours), after which the fermenter was released, followed by solid-liquid separation and subsequent processing. The supernatant or cell mass collected according to the project's R&D requirements (in this experiment, the supernatant was collected).

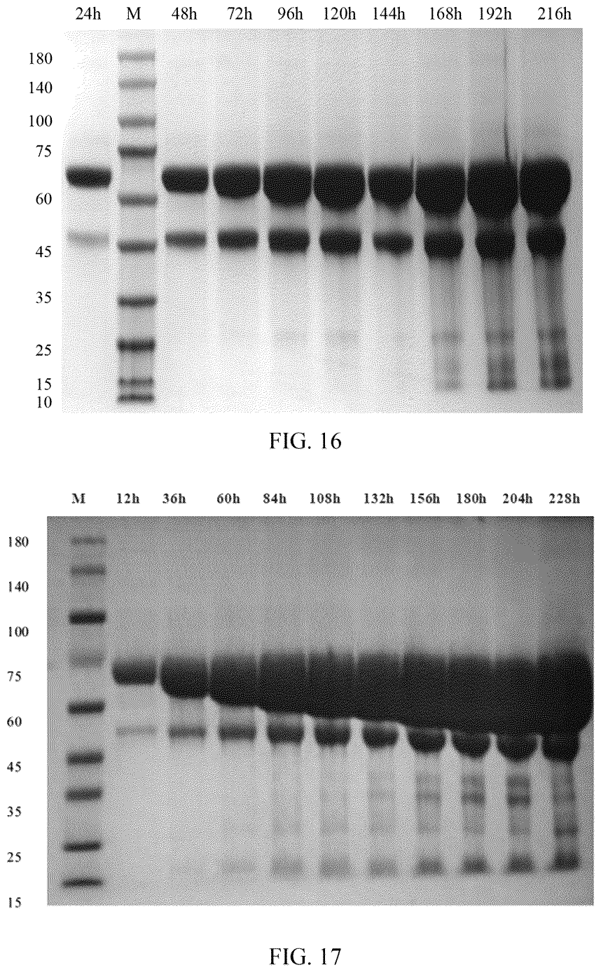

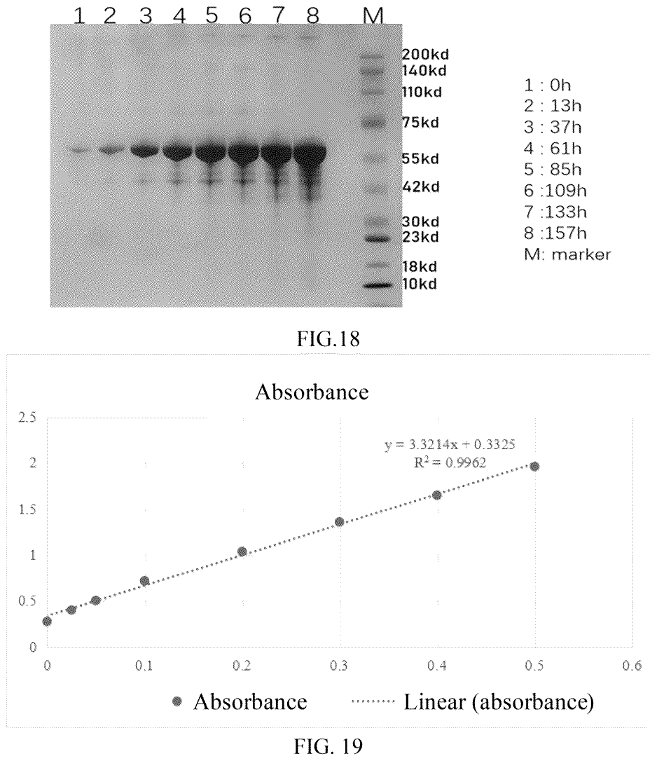

The high-density fermentation results of the engineered Komagataella phaffii strains are shown in FIG. 15, FIG. 16, FIG. 17 and FIG. 18, respectively GS115 (50 L fermenter), CBS7435 (C7) (50 L fermenter), CBS7435(C16) (500 L fermenter), and CBS7435(C16) (2000 L fermenter).

(VII) Detection of Target Protein in the Fermentation Process of Komagataella phaffii Engineered Strains

This invention implements online process control and real-time monitoring for Komagataella phaffii engineered strains during fermentation in bioreactors, with real-time tracking of target protein levels in the fermentation broth at specific induction phases. The detection utilizes Biotechnology's Enhanced BCA Protein Assay Kit, developed based on the BCA method-one of the world's most widely adopted protein concentration assays. This kit delivers high stability, sensitivity, and compatibility in protein concentration measurements. In this experiment, bovine serum albumin (BSA) was used as the standard substance to establish a standard curve and linear equation (see FIG. 19), enabling quantitative analysis of HSA secretion levels in each batch of fermentation broth within the fermenter. The methodology involved sampling the broth at different time points during the induction phase, centrifuging the samples (5000 g×20 min), discarding the cell debris, and retaining the supernatant. After completing the current batch, the retained supernatant samples underwent SDS-PAGE electrophoresis followed by Coomassie Brilliant Blue staining. The protein electrophoresis results were recorded in FIG. 15, FIG. 16, FIG. 17 and FIG. 18. Additionally, the supernatant samples were diluted appropriately, loaded onto 96-well plates with chromogenic reagent, and analyzed using the BCA Protein Concentration Determination Kit (Enhanced Edition) according to the manual. This process enabled calculation of human serum albumin expression levels at various fermentation stages (two batches of fermentation experiments are illustrated in Table 4-5). Furthermore, the expression levels of recombinant HSA from nine additional fermentation batches are presented in Table 6.

TABLE 4
Measured concentrations of the target protein in supernatants collected at different time
points during a 50 L fermenter-based fermentation experiment of the engineered strain C7

| | Induction time | | | | | | | | | | |
|---|---|---|---|---|---|---|---|---|---|---|---|
| | 24 h | 48 h | 72 h | 96 h | 120 h | 144 h | 168 h | 192 h | 216 h | 240 h | 264 h |
| Measured | 0.388 | 0.392 | 0.458 | 0.511 | 0.579 | 0.696 | 0.753 | 0.842 | 0.947 | 1.048 | 1.098 |
| absorbance | | | | | | | | | | | |
| value | | | | | | | | | | | |
| Protein | 1.67 | 1.79 | 3.78 | 5.37 | 7.42 | 10.94 | 12.66 | 15.34 | 18.50 | 21.54 | 23.05 |
| concentration | | | | | | | | | | | |
| (g/L) | | | | | | | | | | | |

TABLE 5
Measured concentrations of the target protein in supernatants collected at different time
points during a 50 L fermenter-based fermentation experiment of the engineered strain C1

| | Induction time | | | | | | | | | | |
|---|---|---|---|---|---|---|---|---|---|---|---|
| | 24 h | 48 h | 72 h | 96 h | 120 h | 144 h | 168 h | 192 h | 216 h | 240 h | 264 h |
| Measured | 0.340 | 0.384 | 0.463 | 0.539 | 0.644 | 0.743 | 0.710 | 0.749 | 0.821 | 0.915 | 1.036 |
| absorbance | | | | | | | | | | | |
| value | | | | | | | | | | | |
| Protein | 0.23 | 1.55 | 3.93 | 6.22 | 9.38 | 12.36 | 11.37 | 12.54 | 14.71 | 17.54 | 21.18 |
| concentration | | | | | | | | | | | |
| (g/L) | | | | | | | | | | | |

TABLE 6
Results of multi-batch production of recombinant HSA using
a high-density fermentation technology induction time

| | | | Methanol | | HSA |
|---|---|---|---|---|---|
| | | | induction | | expression |
| | | | time | Absorbance | level |
| Batch | Strain | pH | (h) | (A562) | (g/L) |
| 1 | C1 | 5.70 | 264 | 1.036 | 21.18 |
| 2 | C3 | 5.71 | 204 | 0.920 | 17.69 |
| 3 | C6 | 5.70 | 204 | 1.190 | 25.82 |
| 4 | C7 | 5.73 | 264 | 1.086 | 23.05 |
| 5 | C18 | 5.74 | 204 | 0.956 | 18.77 |
| 6 | C20 | 5.72 | 204 | 1.016 | 20.58 |
| 7 | G6 | 5.70 | 180 | 0.583 | 7.54 |
| 8 | G15 | 5.71 | 180 | 0.616 | 5.58 |
| 9 | G21 | 5.73 | 180 | 0.525 | 5.80 |

In Table 6, “C” represents the engineered strain CBS7435 and “G” represents the engineered strain GS115.

### Example 7

Study on the Separation and Purification Process of the Fermentation Product of Komagataella phaffii Engineering Strain and the Confirmation of its Structure

In Example 6, the engineered Komagataella phaffii strains were cultured at high cell density using fed-batch fermentation in 50 L, 500 L, and 2000 L fermenters to optimize the BSM (Solution B medium) fermentation process. This study validated the pilot-scale fermentation processes for the GS115 and CBS7435 engineered strains, while also investigating the technical routes for isolating and purifying target products in large-scale fermentation, along with mass spectrometry characterization methods for high-purity samples.

(I) Main Process Steps of Fermentation Products of Komagataella phaffii Engineered Strains

### 1. Fermentation Broth Pretreatment

(1) The fermentation broth is centrifuged at 9000 rpm, with the temperature controlled below 30° C.; (2) Membrane filtration: The supernatant of the fermentation is treated with hollow fiber membrane filtration; (3) Thermal treatment of the fermentation supernatant: In this embodiment, the supernatant from the centrifugation step of the fermentation broth is filtered through a 0.22 m membrane (produced by MILLIPORE); then sodium caprylate is added to achieve a final concentration of 5 mmol/L, the pH is adjusted to 5.8-7.0, and heated at 68° C. for 30 minutes; rapid cooling is performed, followed by pH adjustment to 4.5 using acetic acid.

(4) Membrane filtration: The fermentation supernatant was filtered through hollow fiber membrane after heat treatment, and the sample was purified by column chromatography.

### 2. Column Chromatography of Fermentation Supernatant

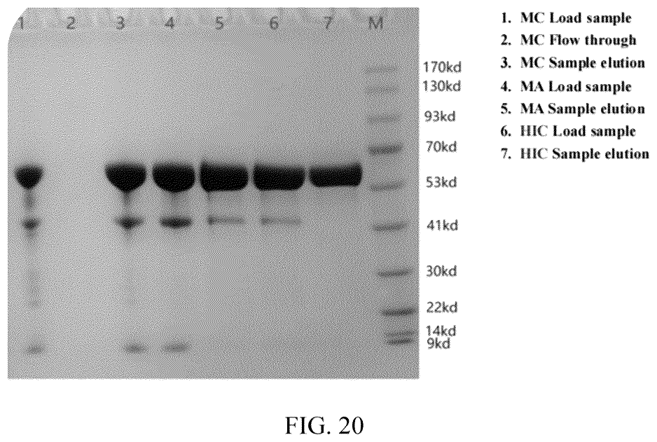

In this embodiment, after extensive screening of purification media, we ultimately selected a novel cationic medium, a novel hydroxyapatite type II chromatography medium, a novel anionic medium, or a hydrophobic chromatography medium. By leveraging the process characteristics of this invention, we optimized the purification steps, enabling the fermentation supernatant to undergo chromatography directly without dilution or desalination, thereby shortening the process cycle. Through repeated research, we established the protein purification protocol: after solid-liquid separation of the fermentation broth, the supernatant was heated to inactivate proteases, followed by hollow fiber filtration, TH-MC composite mode SP cationic chromatography, hydroxyapatite type II (HAPII) chromatography, DEAE anion exchange chromatography, or TA-phenyl-HIC hydrophobic chromatography for further sample purification. The gel electrophoresis showed a single band (see FIG. 20).

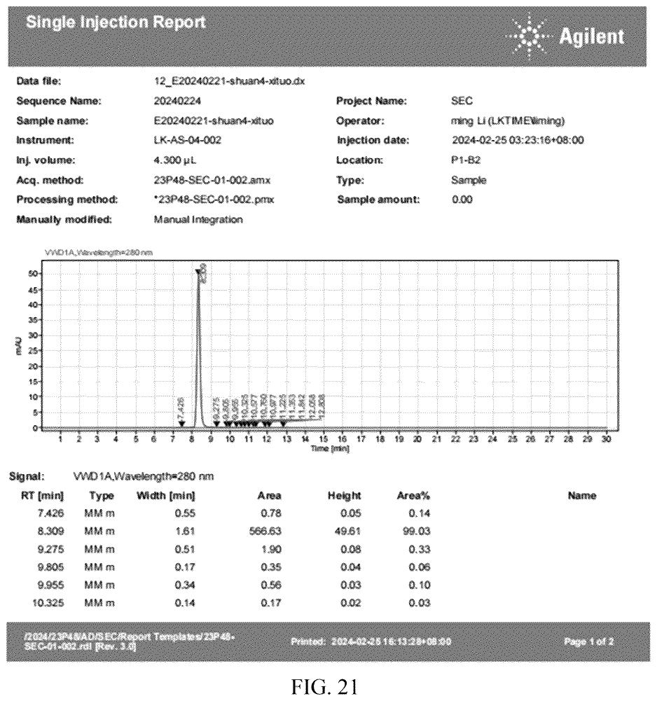

In conclusion, after the above steps 1 and 2, the purity of the recombinant human serum albumin sample was determined by HPLC liquid chromatography analysis, which showed a single peak purity of 99.03%, as shown in FIG. 21.

(II) Determination of the Molecular Weight of rHSA Expressed by Komagataella phaffii

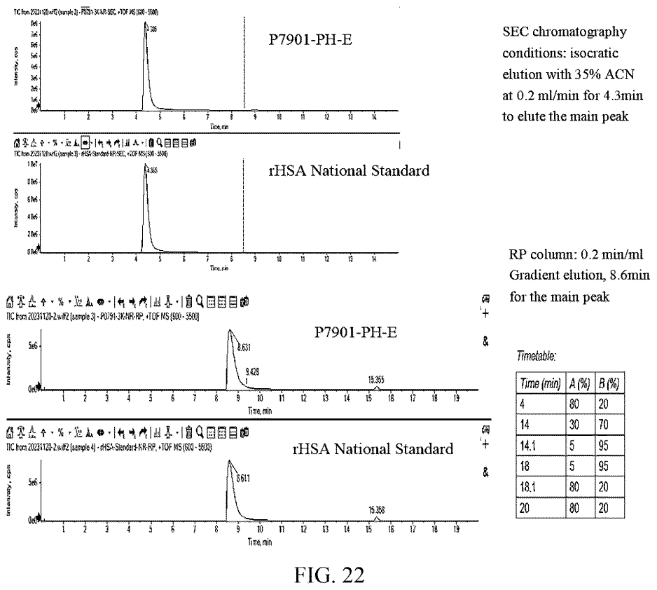

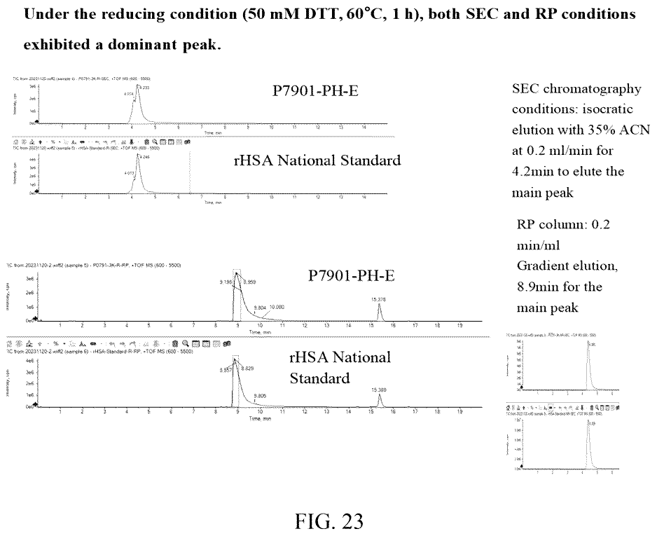

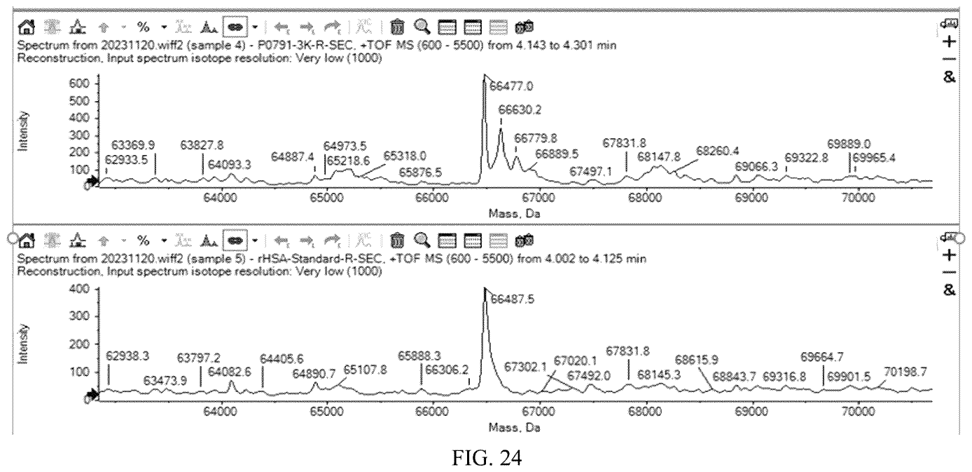

In this embodiment, preliminary research on mass spectrometry molecular weight characterization of the recombinant human serum albumin sample (P7901-PH-E) obtained after separation and purification was conducted with the rHSA national standard (purchased from the National Institutes for Food and Drug Control, China). Comparative studies were performed under non-reducing and reducing conditions using mass spectrometry-SEC and RP elution conditions. The results showed that the total ion chromatogram (TIC) under these conditions exhibited a single peak, with both the peak time and shape showing high consistency (see FIGS. 22-24). Further experiments using electrospray mass spectrometry to determine molecular weight revealed that the recombinant human serum albumin sample (P7901-PH-E) and the rHSA national standard had molecular weights of 66,477.0 Da and 66,487.5 Da, respectively, which showed high consistency with the theoretical molecular weight of HSA (66,472 Da), as shown in FIG. 24.

In conclusion, the novel host strain construction method for high-level expression of human serum albumin in this invention has the following advantages:

1. Through molecular-level optimization of the target gene's full sequence, we obtained the optimal target gene expressed in Pichia pastoris strain.

2. At the cellular level, we introduced the internationally recognized Pichia pastoris expression system host strain CBS7435 for human serum albumin gene expression. By utilizing the geneticin G418 resistance gene to screen Pichia pastoris transformants containing high-copy numbers of exogenous genes, we constructed a high-performance engineered strain for recombinant human serum albumin production.

3. At the engineering level, we optimized fermentation cultivation processes and reactor process controls to further enhance target protein expression levels.

4. In purification technology, we identified and addressed critical flaws in existing recombinant human serum albumin purification methods. By adopting the latest purification media, techniques, and protocols from domestic and international sources, we streamlined the entire purification process to three-stage column chromatography using only solid-liquid separation, thermal treatment, and ultrafiltration concentration. This approach achieved a purity of over 99% for recombinant human serum albumin, with a total purification recovery rate exceeding 60%.

The embodiments described above represent specific implementations of the present invention, with detailed and specific descriptions. However, this should not be construed as limiting the scope of the patent. It should be noted that skilled persons in the art may make various modifications and improvements without departing from the inventive concept, all of which fall within the scope of protection. Therefore, the scope of protection for this patent shall be determined by the appended claims.

What is claimed is:

1. A method for constructing a host strain capable of highly expressing human serum albumin (HSA), comprising the following steps:
S100, designing an optimized gene encoding the HSA;
S200, constructing a recombinant expression vector shown in SEQ ID NO: 3; and
S300, transforming the recombinant expression vector obtained in the step S200 into a Pichia pastoris host strain through electroporation to produce the host strain.

2. The method for constructing the host strain capable of highly expressing the HSA according to claim 1, wherein for the optimized gene encoding the HSA in the step S100, the nucleotide sequence of the optimized gene is shown in SEQ ID NO: 1 and the amino acid sequence of the HSA is shown in SEQ ID NO: 2.

3. The method for constructing the host strain capable of highly expressing the HSA according to claim 1, wherein in the optimized gene encoding the HSA in the step S100, optimal codons preferred by an alcohol oxidase 1 (AOX1) gene derived from Pichia pastoris are adopted, and a proportion of the optimal codons in total codons of the optimized gene is controlled at 90%.

4. The method for constructing the host strain capable of highly expressing the HSA according to claim 1, wherein three restriction endonuclease sites SalI, HindIII, and XbaI are inserted sequentially in a 5′ to 3′ direction within the optimized gene encoding the HSA in the step S100, such that the optimized gene is divided into four relatively-balanced large fragments.

5. The method for constructing the host strain capable of highly expressing the HSA according to claim 1, wherein in the optimized gene encoding the HSA in the step S100, consecutive G-C base pairs are reduced and A-T base pairs preferred by Pichia pastoris are increased, such that a GC content in the optimized gene is adjusted to 45% to 50%.

6. The method for constructing the host strain capable of highly expressing the HSA according to claim 1, wherein the optimized gene encoding the HSA in the step S100 comprises an expression reading cassette constructed as follows:
inserting a 5′ restriction endonuclease site BamHI into a 5′ regulatory region of an AOX1 gene, ligating the 10-deoxynucleotide oligonucleotide CCAAACGATG as shown in SEQ ID NO: 9 involving a Kozak sequence for eukaryotic genes, ligating a yeast α-mating factor leader peptide sequence comprising 85 amino acids derived from Saccharomyces cerevisiae, and inserting a mature HSA gene between multiple cloning sites EcoRI and NotI of a pPIC9K recombinant expression vector, wherein the 5′ regulatory region is a promoter region, and the Kozak sequence for eukaryotic genes is AXXATG; and
inserting a coding sequence AAAAGA for a dibasic amino acid of lysine and arginine after an enzyme cleavage site EcoRI at a 5′ terminus of the mature HSA gene to obtain the sequence as shown in SEQ ID NO: 4; and inserting a double stop codon TAATAG before an enzyme cleavage site NotI at a 3′ terminus of the mature HSA gene to obtain the sequence as shown in SEQ ID NO: 5.

7. The method for constructing the host strain capable of highly expressing the HSA according to claim 1, wherein the constructing the recombinant expression vector in the step S200 is as follows:
excising the optimized gene encoding the HSA fully chemically synthesized from a cloning vector through EcoRI-NotI double enzyme digestion, cutting a gel band with an expected size off, and recovering a target gene-containing fragment with a DNA recovery kit; and cloning the target gene-containing fragment into a recombinant expression vector undergoing corresponding double enzyme digestion to produce the recombinant expression vector.

8. The method for constructing the host strain capable of highly expressing the HSA according to claim 7, wherein the step S300 is as follows: linearizing the recombinant expression vector obtained in the step S200 with a restriction endonuclease SacI or a restriction endonuclease BglII to obtain a linearized recombinant expression vector, and transforming the linearized recombinant expression vector into the Pichia pastoris host strain through the electroporation to produce the host strain.

9. The method for constructing the host strain capable of highly expressing the HSA according to claim 8, wherein the nucleotide sequence of the recombinant expression vector is shown in SEQ ID NO: 1.

10. The method for constructing the host strain capable of highly expressing the HSA according to claim 7, wherein the recombinant expression vector is one of pPIC9, pPIC3, pPICZαABC, pPIC3.5K, pHIL-S1, pHIL-D2, pA0804, pA0815, pGAPZαABC, pPIC6αABC, and pPIC9K.

11. The method for constructing the host strain capable of highly expressing the HSA according to claim 10, wherein the recombinant expression vector is the pPIC9K.

12. The method for constructing the host strain capable of highly expressing the HSA according to claim 8, wherein the Pichia pastoris host strain is Pichia pastoris CBS7435.

13. The method for constructing the host strain capable of highly expressing the HSA according to claim 1, wherein the host strain is Pichia pastoris HSA-C16, wherein the Pichia pastoris HSA-C16 is deposited at China General Microbiological Culture Collection Center (CGMCC), with an accession number of CGMCC No. 30175, and a deposit date is Mar. 28, 2024.

14. The method for constructing the host strain capable of highly expressing the HSA according to claim 2, wherein the constructing the recombinant expression vector in the step S200 is as follows:
excising the optimized gene encoding the HSA fully chemically synthesized from a cloning vector through EcoRI-NotI double enzyme digestion, cutting a gel band with an expected size off, and recovering a target gene-containing fragment with a DNA recovery kit; and cloning the target gene-containing fragment into a recombinant expression vector undergoing corresponding double enzyme digestion to produce the recombinant expression vector.

15. The method for constructing the host strain capable of highly expressing the HSA according to claim 3, wherein the constructing the recombinant expression vector in the step S200 is as follows:
excising the optimized gene encoding the HSA fully chemically synthesized from a cloning vector through EcoRI-NotI double enzyme digestion, cutting a gel band with an expected size off, and recovering a target gene-containing fragment with a DNA recovery kit; and cloning the target gene-containing fragment into a recombinant expression vector undergoing corresponding double enzyme digestion to produce the recombinant expression vector.

16. The method for constructing the host strain capable of highly expressing the HSA according to claim 4, wherein the constructing the recombinant expression vector in the step S200 is as follows:
excising the optimized gene encoding the HSA fully chemically synthesized from a cloning vector through EcoRI-NotI double enzyme digestion, cutting a gel band with an expected size off, and recovering a target gene-containing fragment with a DNA recovery kit; and cloning the target gene-containing fragment into a recombinant expression vector undergoing corresponding double enzyme digestion to produce the recombinant expression vector.

17. The method for constructing the host strain capable of highly expressing the HSA according to claim 5, wherein the constructing the recombinant expression vector in the step S200 is as follows:
excising the optimized gene encoding the HSA fully chemically synthesized from a cloning vector through EcoRI-NotI double enzyme digestion, cutting a gel band with an expected size off, and recovering a target gene-containing fragment with a DNA recovery kit; and cloning the target gene-containing fragment into a recombinant expression vector undergoing corresponding double enzyme digestion to produce the recombinant expression vector.

18. The method for constructing the host strain capable of highly expressing the HSA according to claim 6, wherein the constructing the recombinant expression vector in the step S200 is as follows:
excising the optimized gene encoding the HSA fully chemically synthesized from a cloning vector through EcoRI-NotI double enzyme digestion, cutting a gel band with an expected size off, and recovering a target gene-containing fragment with a DNA recovery kit; and cloning the target gene-containing fragment into a recombinant expression vector undergoing corresponding double enzyme digestion to produce the recombinant expression vector.
